(19) United States
(12) Patent Application Publication
(10) Pub. No.: US 2009/0054235 A1
(43) Pub. Date: Feb. 26, 2009

(54) Benzoheterocyclylethylbenzamide derivatives

(76) Inventors: Darren Mansfield, Bergisch Gladbach (DE); Pierre-Yves Coqueron, Lyon (FR); Heiko Rieck, Burscheid (DE); Philippe Desbordes, Lyon (FR); Alain Villier, Collonges au Mont d'Or (FR); Marie-Claire Grosjean-Cournoyer, Curis au Mont d'Or (FR); Pierre Genix, Lyon (FR)

Correspondence Address: OSTROLENK FABER GERB & SOFFEN, 1180 AVENUE OF THE AMERICAS, NEW YORK, NY, 100368403, US

(21) Appl. No.: 11/988,611

(22) PCT Filed: Jul. 6, 2006
(86) PCT No.: PCT/EP2006/063987

§ 371 Date: Apr. 17, 2008

(30) Foreign Application Priority Data
Jul. 12, 2005 (EP) 05356116.3

(51) Int. Cl. A01C 1/06; C07D 215/38; A61K 31/47; C07D 235/00; A61K 31/4164; C07D 333/50; A61K 31/381; C07D 307/00; A61K 31/343; C07D 209/04; A61K 31/40

(52) U.S. Cl. 504/100; 546/175; 514/311; 548/304.4; 514/394; 549/58; 514/443; 549/467; 514/469; 548/491; 514/419

(57) ABSTRACT

The present invention relates to novel benzoheterocyclylethylbenzamide derivatives of formula (I) in which the substituents are in the description, their process of preparation, their use as fungicides, particularly in the form of fungicidal compositions, and methods for the control of phytopathogenic fungi of plants using these compounds or their compositions.

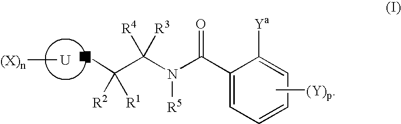

The present invention relates to novel benzoheterocyclylethylbenzamide derivatives, their process of preparation, their use as fungicides, particularly in the form of fungicidal compositions, and methods for the control of phytopathogenic fungi of plants using these compounds or their compositions.

In the Chinese patent application CN 1365971, certain benzoheterocyclylethylbenzamide derivatives in which the benzoheterocycle is the 3-1H-2H-indolyl are generically embraced in a broad disclosure of numerous N-acyltryptamine compounds. However, the said compounds are not included in the benzoheterocyclylethylbenzamide derivatives concerned by the present invention, and in addition, this document does not specifically disclose nor suggest to select such compounds.

It is always of high-interest in the field of agrochemicals to use novel pesticidal compounds in order to avoid or to fight the development of resistant strains to the active ingredients used by the farmer.

We have now found a new family of compounds which possess the above mentioned characteristics.

Accordingly, the present invention relates to a benzo-heterocyclylethylbenzamide derivative of general formula (I)

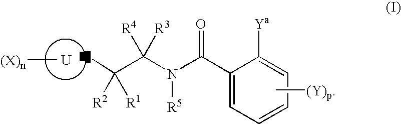

in which:

n is 0, 1, 2, 3, 4, 5 or 6;

p is 1, 2, 3 or 4;

U is:

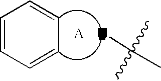

wherein A represents a 5- or 6-membered heterocycle with one, two or three heteroatoms which may be the same or different; fused with a phenyl ring to form a benzoheterocycle provided that this benzoheterocycle is different from 3-1H-2H-indolyl

Z is a carbon atom or a heteroatom which can not be substituted by X;

X is the same or different and is a halogen atom, a nitro group, a cyano group, a hydroxy group, an amino group, a sulfanyl group, a pentafluoro-λ⁶-sulfanyl group, a formyl group, a formyloxy group, a formylamino group, a carboxy group, a carbamoyl group, a N-hydroxycarbamoyl group, a carbamate group, a (hydroxyimino)-C₁-C₆-alkyl group, a C₁-C₈-alkyl, a C₂-C₈-alkenyl, a C₂-C₈-alkynyl, a C₁-C₈-alkylamino, a C₁-C₈-halogenoalkyl having 1 to 5 halogen atoms, a di-C₁-C₈-alkylamino, a C₁-C₈-alkoxy, a C₁-C₈-halogenoalkoxy having 1 to 5 halogen atoms, a C₁-C₈-alkylsulfanyl, a C₁-C₈-halogenoalkylsulfanyl having 1 to 5 halogen atoms, a C₂-C₈-alkenyloxy, a C₂-C₈-halogenoalkenyloxy having 1 to 5 halogen atoms, a C₃-C₈-alkynyloxy, a C₃-C₈-halogenoalkynyloxy having 1 to 5 halogen atoms, a C₃-C₈-cycloalkyl, a C₃-C₈-halogenocycloalkyl having 1 to 5 halogen atoms, a C₁-C₈-alkylcarbonyl, a C₁-C₈-halogenoalkylcarbonyl having 1 to 5 halogen atoms, a C₁-C₈-alkylcarbamoyl, a di-C₁-C₈-alkylcarbamoyl, a N—C₁-C₈-alkyloxycarbamoyl, a C₁-C₈-alkoxycarbamoyl, a N—C₁-C₈-alkyl-C₁-C₈-alkoxycarbamoyl, a C₁-C₈-alkoxycarbonyl, a C₁-C₈-halogenoalkoxycarbonyl having 1 to 5 halogen atoms, a C₁-C₈-alkylcarbonyloxy, a C₁-C₈-halogenoalkylcarbonyloxy having 1 to 5 halogen atoms, a C₁-C₈-alkylcarbonylamino, a C₁-C₈-halogenoalkylcarbonylamino having 1 to 5 halogen atoms, a C₁-C₈-alkylaminocarbonyloxy, a di-C₁-C₈-alkylaminocarbonyloxy, a C₁-C₈-alkyloxycarbonyloxy, a C₁-C₈-alkylsulphenyl, a C₁-C₈-halogenoalkylsulphenyl having 1 to 5 halogen atoms, a C₁-C₈-alkylsulphinyl, a C₁-C₈-halogenoalkylsulphinyl having 1 to 5 halogen atoms, a C₁-C₈-alkylsulphonyl, a C₁-C₈-halogenoalkylsulphonyl having 1 to 5 halogen atoms, a C₁-C₆-alkoxyimino, a (C₁-C₆-alkoxyimino)-C₁-C₆-alkyl, a (C₁-C₆-alkenyloxyimino)-C₁-C₆-alkyl, a (C₁-C₆-alkynyloxyimino)-C₁-C₆-alkyl, a (benzyloxyimino)-C₁-C₆-alkyl, a benzyloxy, a benzylsulfanyl, a benzylamino, a phenoxy, a phenylsulfanyl a phenyl or a phenylamino;
R¹ and R² are the same or different and are a hydrogen atom, a cyano group, a formyl group, a carboxy group, a carbamoyl group, a (hydroxyimino)-C₁-C₆-alkyl group, a C₁-C₈-alkyl, a C₂-C₈-alkenyl, a C₂-C₈-alkynyl, a C₃-C₈-cycloalkyl, a C₃-C₈-halogenocycloalkyl having 1 to 5 halogen atoms, a C₁-C₈-alkylcarbonyl, a C₁-C₈-halogenoalkylcarbonyl having 1 to 5 halogen atoms, a C₁-C₈-halogenoalkyl having 1 to 5 halogen atoms, a C₁-C₈-alkylcarbamoyl, a di-C₁-C₈-alkylcarbamoyl, a C₁-C₈-alkoxycarbamoyl, a C₁-C₈-alkoxycarbonyl or a C₁-C₈-halogenoalkoxycarbonyl having 1 to 5 halogen atoms;

R³ and R⁴ are the same or different and are a hydrogen atom, a formyl group, a carbamoyl group, a (hydroxyimino)-C₁-C₆-alkyl group, a C₁-C₈-alkyl, a C₂-C₈-alkenyl, a C₂-C₈-alkynyl, a C₃-C₈-cycloalkyl, a C₃-C₈-halogenocycloalkyl having 1 to 5 halogen atoms, a C₁-C₈-alkylcarbonyl, a C₁-C₈-halogenoalkylcarbonyl having 1 to 5 halogen atoms, a C₁-C₈-halogenoalkyl having 1 to 5 halogen atoms, a C₁-C₈-alkylcarbamoyl, a di-C₁-C₈-alkylcarbamoyl or a C₁-C₈-alkoxycarbamoyl;

R⁵ is a hydrogen atom, a C₁-C₆-alkyl, or a C₃-C₇-cycloalkyl;

Y is the same or different and is a hydrogen atom, a halogen atom, a nitro group, a cyano group, a hydroxy group, an amino group, a sulfanyl group, a pentafluoro-λ⁶-sulfanyl group, a formyl group, a formyloxy group, a formylamino group, a carboxy group, a carbamoyl group, a N-hydroxycarbamoyl group, a carbamate group, a (hydroxyimino)-C₁-C₆-alkyl group, a C₁-C₈-alkyl, a C₂-C₈-alkenyl, a C₂-C₈-alkynyl, a C₁-C₈-alkylamino, a di-C₁-C₈-alkylamino, a C₁-C₈-alkoxy, a C₁-C₈-halogenoalkyl having 1 to 5 halogen atoms, a C₁-C₈-halogenoalkoxy having 1 to 5 halogen atoms, a C₁-C₈-alkylsulfanyl, a C₁-C₈-halogenoalkylsulfanyl having 1 to 5 halogen atoms, a C₂-C₈-alkenyloxy, a C₂-C₈-halogenoalkenyloxy having 1 to 5 halogen atoms, a C₃-C₈-alkynyloxy, a C₃-C₈-halogenoalkynyloxy having 1 to 5 halogen atoms, a C₃-C₈-cycloalkyl, a C₃-C₈-halogenocycloalkyl having 1 to 5 halogen atoms, a C₁-C₈-alkylcarbonyl, a C₁-C₈-halogenoalkylcarbonyl having 1 to 5 halogen atoms, a C₁-C₈-alkylcarbamoyl, a di-C₁-C₈-alkylcarbamoyl, a N—C₁-C₈-alkyloxycarbamoyl, a C₁-C₈-alkoxycarbamoyl, a N—C₁-C₈-alkyl-C₁-C₈-alkoxycarbamoyl, a C₁-C₈-alkoxycarbonyl, a C₁-C₈-halogenoalkoxycarbonyl having 1 to 5 halogen atoms, a C₁-C₈-alkylcarbonyloxy, a C₁-C₈-halogenoalkylcarbonyloxy having 1 to 5 halogen atoms, a C₁-C₈-alkylcarbonylamino, a C₁-C₈-halogenoalkylcarbonylamino having 1 to 5 halogen atoms, a C₁-C₈-alkylaminocarbonyloxy, a di-C₁-C₈-alkylaminocarbonyloxy, a C₁-C₈-alkyloxycarbonyloxy, a C₁-C₈-alkylsulphenyl, a C₁-C₈-halogenoalkylsulphenyl having 1 to 5 halogen atoms, a C₁-C₈-alkylsulphinyl, a C₁-C₈-halogenoalkylsulphinyl having 1 to 5 halogen atoms, a C₁-C₈-alkylsulphonyl, a C₁-C₈-halogenoalkyl-sulphonyl having 1 to 5 halogen atoms, a C₁-C₆-alkoxyimino, a (C₁-C₆-alkoxyimino)-C₁-C₆-alkyl, a (C₁-C₆-alkenyloxyimino)-C₁-C₆-alkyl, a (C₁-C₆-alkynyloxyimino)-C₁-C₆-alkyl or a (benzyloxyimino)-C₁-C₆-alkyl; and

Y^{a} is a halogen atom, a cyano group, a sulfanyl group, a pentafluoro-λ⁶-sulfanyl group, a formyl group, a formyloxy group, a formylamino group, a carbamoyl group, a N-hydroxycarbamoyl group, a carbamate group, a (hydroxyimino)-C₁-C₆-alkyl group, a C₁-C₈-alkyl, a C₂-C₈-alkenyl, a C₂-C₈-alkynyl, a C₁-C₈-alkoxy, a C₁-C₈-halogenoalkyl having 1 to 5 halogen atoms, a C₁-C₈-halogenoalkoxy having 1 to 5 halogen atoms, a C₁-C₈-alkylsulfanyl, a C₁-C₈-halogenoalkylsulfanyl having 1 to 5 halogen atoms, a C₂-C₈-alkenyloxy, a C₂-C₈-halogenoalkenyloxy having 1 to 5 halogen atoms, a C₃-C₈-alkynyloxy, a C₃-C₈-halogenoalkynyloxy having 1 to 5 halogen atoms, a C₃-C₈-cycloalkyl, a C₃-C₈-halogenocycloalkyl having 1 to 5 halogen atoms, a C₁-C₈-alkylcarbonyl, a C₁-C₈-halogenoalkylcarbonyl having 1 to 5 halogen atoms, a C₁-C₈-alkylcarbamoyl, a di-C₁-C₈-alkylcarbamoyl, a N—C₁-C₈-alkyloxycarbamoyl, a C₁-C₈-alkoxycarbamoyl, a N—C₁-C₈-alkyl-C₁-C₈-alkoxycarbamoyl, a C₁-C₈-alkoxycarbonyl, a C₁-C₈-halogenoalkoxycarbonyl having 1 to 5 halogen atoms, a C₁-C₈-alkylcarbonyloxy, a C₁-C₈-halogenoalkylcarbonyloxy having 1 to 5 halogen atoms, a C₁-C₈-alkylcarbonylamino, a C₁-C₈-halogenoalkylcarbonylamino having 1 to 5 halogen atoms, a C₁-C₈-alkylaminocarbonyloxy, a di-C₁-C₈-alkylaminocarbonyloxy, a C₁-C₈-alkyloxycarbonyloxy, a C₁-C₈-alkylsulphenyl, a C₁-C₈-halogenoalkylsulphenyl having 1 to 5 halogen atoms, a C₁-C₈-alkylsulphinyl, a C₁-C₈-halogenoalkylsulphinyl having 1 to 5 halogen atoms, a C₁-C₈-alkylsulphonyl, a C₁-C₈-halogenoalkyl-sulphonyl having 1 to 5 halogen atoms, a C₁-C₆-alkoxyimino, a (C₁-C₆-alkoxyimino)-C₁-C₆-alkyl, a (C₁-C₆-alkenyloxyimino)-C₁-C₆-alkyl or a (C₁-C₆-alkynyloxyimino)-C₁-C₆-alkyl, a (benzyloxyimino)-C₁-C₆-alkyl;

as well as its salts, N-oxydes, metallic complexes, metalloidic complexes and optically active isomers.

In an embodiment of the present invention, A represents a 5- or 6-membered heterocycle with one, two or three heteroatoms which may be the same or different; fused with a phenyl ring to form a benzoheterocycle provided that this benzoheterocycle is different from 3-2H-indolyl, then Z, X, R¹, R², R³, R⁴, R⁵, y, Y^{a} are as defined above.

In the context of the present invention:

halogen means fluorine, bromine, chlorine or iodine.

carboxy means —C(═O)OH; carbonyl means —C(═O)—; carbamoyl means —C(═O)NH₂; N-hydroxycarbamoyl means —C(═O)NHOH;

an alkyl group, an alkenyl group, and an alkynyl group as well as moieties containing these terms, can be linear or branched; and

heteroatom means sulphur, nitrogen or oxygen.

In the context of the present invention, it has also to be understood that in the case of di-substituted amino and of di-substituted carbamoyl radicals, the two substituents may form together with the nitrogen atom bearing them a saturated heterocyclic ring containing 3 to 7 atoms.

Any of the compounds of the present invention can exist in one or more optical or chiral isomer forms depending on the number of asymmetric centres in the compound. The invention thus relates equally to all the optical isomers and to their racemic or scalemic mixtures (the term “scalemic” denotes a mixture of enantiomers in different proportions), and to the mixtures of all the possible stereoisomers, in all proportions. The diastereoisomers and/or the optical isomers can be separated according to the methods which are known per se by the man ordinary skilled in the art.

Any of the compounds of the present invention can also exist in one or more geometric isomer forms depending on the number of double bonds in the compound. The invention thus relates equally to all geometric isomers and to all possible mixtures, in all proportions. The geometric isomers can be separated according to general methods, which are known per se by the man ordinary skilled in the art.

Any of the compounds of general formula (I) wherein X represents a hydroxy, a sulfanyl group or an amino group may be found in its tautomeric form resulting from the shift of the proton of said hydroxy, sulfanyl or amino group. Such tautomeric forms of such compounds are also part of the present invention. More generally speaking, all tautomeric forms of compounds of general formula (I) wherein X represents a hydroxy, a sulfanyl group or an amino group, as well as the tautomeric forms of the compounds which can optionally be used as intermediates in the preparation processes, and which will be defined in the description of these processes, are also part of the present invention.

According to the present invention, the “U” group may be substituted in any position by (X)ₙ, in which X and n are as defined above. Preferably, the present invention relates to a heterocyclylethylbenzamide derivative of general formula (I) in which the different characteristics may be chosen alone or in combination as being:

as regards n, n is 0, 1 or 2; and

as regards X, X is chosen as being a C₁-C₄-alkyl group, a halogen atom, a hydroxyl group, an amino group, a nitro group, a C₁-C₄-alkoxy group, a C₁-C₄-alkylthio, a C₁-C₄-alkylsulphonyl, a C₁-C₄-halogenoalkyl having 1 to 5 halogen atoms.

According to the present invention, the carbon atoms of the carboxamide moiety of the compound of formula (I) are substituted by R¹, R², R³ and R⁴; R¹, R², R³ and R⁴ being as defined above. Preferably, the present invention also relates to a benzoheterocyclylethylbenzamide derivative of general formula (I) in which the different characteristics may be chosen alone or in combination as being:

as regards R¹ and R², R¹ and R² are chosen, independently of each other, as being a hydrogen atom, a C₁-C₈-alkyl, a C₂-C₈-alkenyl, a C₂-C₈-alkynyl, a C₃-C₈-cycloalkyl, a C₃-C₈-halogenocycloalkyl having 1 to 5 halogen atoms, a C₁-C₈-halogenoalkyl having 1 to 5 halogen atoms; and

as regards R³ and R⁴, R³ and R⁴ are chosen, independently of each other, as being a hydrogen atom, a C₁-C₈-alkyl, a C₂-C₈-alkenyl, a C₂-C₈-alkynyl, a C₃-C₈-cycloalkyl, a C₃-C₈-halogenocycloalkyl having 1 to 5 halogen atoms, a C₁-C₈-halogenoalkyl having 1 to 5 halogen atoms.

According to the present invention, the nitrogen atom of the carboxamide moiety of the compound of formula (I) is substituted by R⁵, R⁵ being a hydrogen atom, a C₁-C₆-alkyl or a C₃-C₇-cycloalkyl. Preferably, the C₃-C₇-cycloalkyl is cyclopropyl.

According to the present invention, the phenyl moiety is substituted in the ortho position by Y^{a} and may be substituted in any other position by (Y)ₚ, in which Y^{a}, y and p are defined as above. Preferably, the present invention relates to a benzoheterocyclylethylbenzamide derivative of the general formula (I) in which the different characteristics may be chosen alone or in combination as being:

as regards Y^{a}, Y^{a} is chosen as being a halogen atom, a pentafluoro-λ⁶-sulfanyl group, a C₁-C₈-alkyl, a C₁-C₈-halogenoalkyl having 1 to 5 halogen atoms or a C₁-C₈-halogenoalkoxy having 1 to 5 halogen atoms;

as regards p, p is 1;

as regards Y, Y is chosen as being a hydrogen atom or a halogen atom, more preferably Y is a hydrogen atom.

According to the present invention, the “U” group of the compound of general formula (I) is a benzoheterocycle where the heterocycle fused to the phenyl ring is a five or six membered ring with one, two or three heteroatoms which may be the same or different. Preferably, the present invention also relates to a benzoheterocyclylethylbenzamide derivative of general formula (I) in which the different characteristics may be chosen alone or in combination as being:

U is chosen as being a 2-benzothiophene, a 3-benzothiophene, a 1-indole, a 2-indole, a 3-indole provided it is substituted in the 1- or in the 2-position, a 2-benzofuran, a 3-benzofuran, a 2-benzoxazole, a 2-benzothiazole, a 1-benzimidazole, a 2-benzimidazole, a 3-benz-1,2-isoxazole, a 3-benz-2,1-isoxazole, a 3-benz-1,2-isothiazole, a 3-benz-2,1-isothiazole, a 1-benzopyrazole, a 2-benzopyrazole, a 3-benzopyrazole, a 2-quinoline, a 3-quinoline, a 4-quinoline, a 1-isoquinoline, a 3-isoquinoline, a 4-isoquinoline.

U is non substituted or substituted by 1, 2, 3, 4, 5, or 6 groups which are chosen, independently of each other, as being a halogen atom, a hydroxyl group, an amino group, a nitro group, a C₁-C₄-alkyl, a C₁-C₄-alkoxy group, a C₁-C₄-alkylthio, a C₁-C₄-alkylsulphonyl, a C₁-C₄-halogenoalkyl having 1 to 5 halogen atoms.

Specific examples of “U” group include:

U represents a benzoheterocycle of the general formula (U-1)

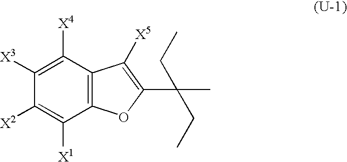

in which:

X¹, X², X³, X⁴ and X⁵ may be the same or different and may be a hydrogen atom, a halogen atom, a hydroxyl group, an amino group, a nitro group, a C₁-C₄-alkyl, a C₁-C₄-alkoxy group, a C₁-C₄-alkylthio, a C₁-C₄-alkylsulphonyl or a C₁-C₄-halogenoalkyl having 1 to 5 halogen atoms

U represents a benzoheterocycle of the general formula (U-2)

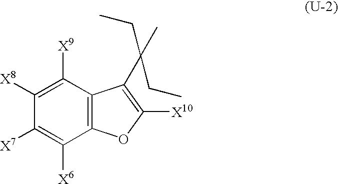

in which:

X⁶, X⁷, X⁸, X⁹ and X¹⁰ may be the same or different and may be a hydrogen atom, a halogen atom, a hydroxyl group, an amino group, a nitro group, a C₁-C₄-alkyl, a C₁-C₄-alkoxy group, a C₁-C₄-alkylthio, a C₁-C₄-alkylsulphonyl or a C₁-C₄-halogenoalkyl having 1 to 5 halogen atoms.

U represents a benzoheterocycle of the general formula (U-3)

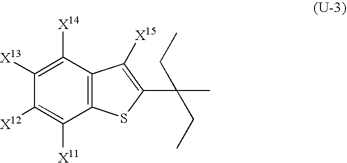

in which:

X¹¹, X¹², X¹³, X¹⁴ and X¹⁵ may be the same or different and may be a hydrogen atom, a halogen atom, a hydroxyl group, an amino group, a nitro group, a C₁-C₄-alkyl, a C₁-C₄-alkoxy group, a C₁-C₄-alkylthio, a C₁-C₄-alkylsulphonyl or a C₁-C₄-halogenoalkyl having 1 to 5 halogen atoms

U represents a benzoheterocycle of the general formula (U-4)

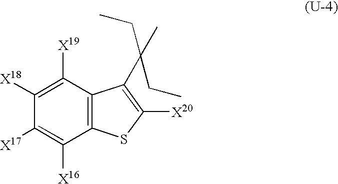

in which:

X¹⁶, X¹⁷, X¹⁸, X¹⁹ and X²⁰ may be the same or different and may be a hydrogen atom, a halogen atom, a hydroxyl group, an amino group, a nitro group, a C₁-C₄-alkyl, a C₁-C₄-alkoxy group, a C₁-C₄-alkylthio, a C₁-C₄-alkylsulphonyl or a C₁-C₄-halogenoalkyl having 1 to 5 halogen atoms.

U represents a benzoheterocycle of the general formula (U-5)

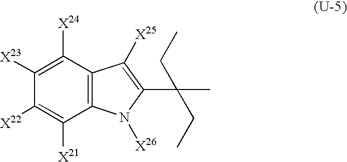

in which:

X²¹, X²², X²³, X²⁴, X²⁵ and X²⁶ may be the same or different and may be a hydrogen atom, a halogen atom, a hydroxyl group, an amino group, a nitro group, a C₁-C₄-alkyl, a C₁-C₄-alkoxy group, a C₁-C₄-alkylthio, a C₁-C₄-alkylsulphonyl or a C₁-C₄-halogenoalkyl having 1 to 5 halogen atoms

U represents a benzoheterocycle of the general formula (U-6)

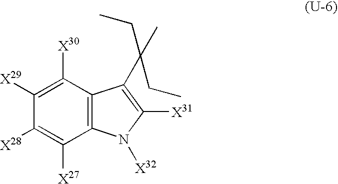

in which:

X²⁷, X²⁸, X²⁹, X³⁰, X³¹ and X³² may be the same or different and may be a hydrogen atom, a halogen atom, a hydroxyl group, an amino group, a nitro group, a C₁-C₄-alkyl, a C₁-C₄-alkoxy group, a C₁-C₄-alkylthio, a C₁-C₄-alkylsulphonyl or a C₁-C₄-halogenoalkyl having 1 to 5 halogen atoms;

provided that X³¹ and X³² are not both a hydrogen atom

U represents a benzoheterocycle of the general formula (U-7)

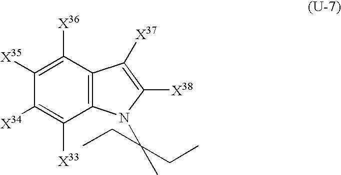

in which:

X³³, X³⁴, X³⁵, X³⁶, X³⁷ and X³⁸ may be the same or different and may be a hydrogen atom, a halogen atom, a hydroxyl group, an amino group, a nitro group, a C₁-C₄-alkyl, a C₁-C₄-alkoxy group, a C₁-C₄-alkylthio, a C₁-C₄-alkylsulphonyl or a C₁-C₄-halogenoalkyl having 1 to 5 halogen atoms.

U represents a benzoheterocycle of the general formula (U-8)

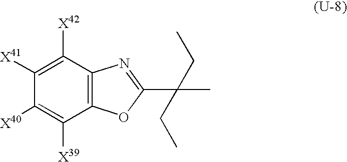

in which:

X³⁹, X⁴⁰, X⁴¹ and X⁴² may be the same or different and may be a hydrogen atom, a halogen atom, a hydroxyl group, an amino group, a nitro group, a C₁-C₄-alkyl, a C₁-C₄-alkoxy group, a C₁-C₄-alkylthio, a C₁-C₄-alkylsulphonyl or a C₁-C₄-halogenoalkyl having 1 to 5 halogen atoms.

U represents a benzoheterocycle of the general formula (U-9)

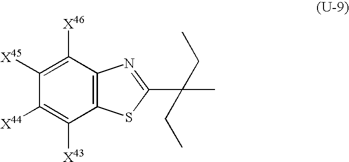

in which:

X⁴³, X⁴⁴, X⁴⁵ and X⁴⁶ may be the same or different and may be a hydrogen atom, a halogen atom, a hydroxyl group, an amino group, a nitro group, a C₁-C₄-alkyl, a C₁-C₄-alkoxy group, a C₁-C₄-alkylthio, a C₁-C₄-alkylsulphonyl or a C₁-C₄-halogenoalkyl having 1 to 5 halogen atoms.

U represents a benzoheterocycle of the general formula (U-10)

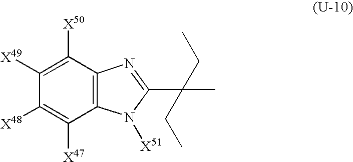

in which:

X⁴⁷, X⁴⁸, X⁴⁹, X⁵⁰ and X⁵¹ may be the same or different and may be a hydrogen atom, a halogen atom, a hydroxyl group, an amino group, a nitro group, a C₁-C₄-alkyl, a C₁-C₄-alkoxy group, a C₁-C₄-alkylthio, a C₁-C₄-alkylsulphonyl or a C₁-C₄-halogenoalkyl having 1 to 5 halogen atoms.

U represents a benzoheterocycle of the general formula (U-11)

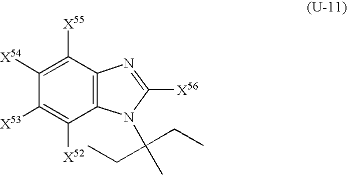

in which:

X⁵², X⁵³, X⁵⁴, X⁵⁵ and X⁵⁶ may be the same or different and may be a hydrogen atom, a halogen atom, a hydroxyl group, an amino group, a nitro group, a C₁-C₄-alkyl, a C₁-C₄-alkoxy group, a C₁-C₄-alkylthio, a C₁-C₄-alkylsulphonyl or a C₁-C₄-halogenoalkyl having 1 to 5 halogen atoms.

U represents a benzoheterocycle of the general formula (U-12)

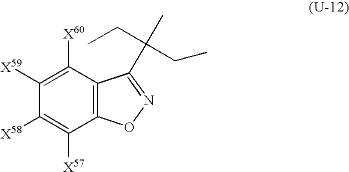

in which:

X⁵⁷, X⁵⁸, X⁵⁹ and X⁶⁰ may be the same or different and may be a hydrogen atom, a halogen atom, a hydroxyl group, an amino group, a nitro group, a C₁-C₄-alkyl, a C₁-C₄-alkoxy group, a C₁-C₄-alkylthio, a C₁-C₄-alkylsulphonyl or a C₁-C₄-halogenoalkyl having 1 to 5 halogen atoms.

U represents a benzoheterocycle of the general formula (U-13)

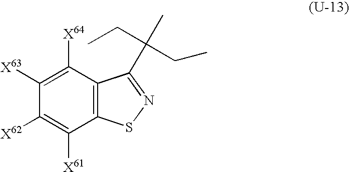

in which:

X⁶¹, X⁶², X⁶³ and X⁶⁴ may be the same or different and may be a hydrogen atom, a halogen atom, a hydroxyl group, an amino group, a nitro group, a C₁-C₄-alkyl, a C₁-C₄-alkoxy group, a C₁-C₄-alkylthio, a C₁-C₄-alkylsulphonyl or a C₁-C₄-halogenoalkyl having 1 to 5 halogen atoms.

U represents a benzoheterocycle of the general formula (U-14)

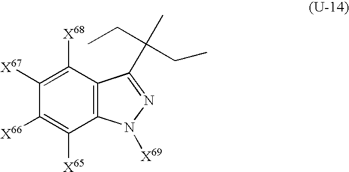

in which:

X⁶⁵, X⁶⁶, X⁶⁷, X⁶⁸ and X⁶⁹ may be the same or different and may be a hydrogen atom, a halogen atom, a hydroxyl group, an amino group, a nitro group, a C₁-C₄-alkyl, a C₁-C₄-alkoxy group, a C₁-C₄-alkylthio, a C₁-C₄-alkylsulphonyl or a C₁-C₄-halogenoalkyl having 1 to 5 halogen atoms.

U represents a benzoheterocycle of the general formula (U-15)

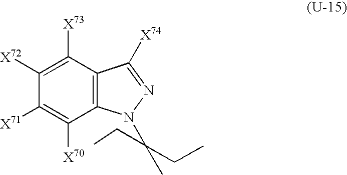

in which:

X⁷⁰, X⁷¹, X⁷², X⁷³ and X⁷⁴ may be the same or different and may be a hydrogen atom, a halogen atom, a hydroxyl group, an amino group, a nitro group, a C₁-C₄-alkyl, a C₁-C₄-alkoxy group, a C₁-C₄-alkylthio, a C₁-C₄-alkylsulphonyl or a C₁-C₄-halogenoalkyl having 1 to 5 halogen atoms.

U represents a benzoheterocycle of the general formula (U-16)

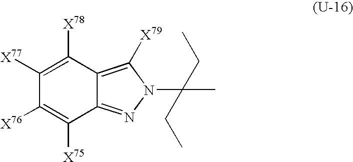

in which:

X⁷⁵, X⁷⁶, X⁷⁷, X⁷⁸ and X⁷⁹ may be the same or different and may be a hydrogen atom, a halogen atom, a hydroxyl group, an amino group, a nitro group, a C₁-C₄-alkyl, a C₁-C₄-alkoxy group, a C₁-C₄-alkylthio, a C₁-C₄-alkylsulphonyl or a C₁-C₄-halogenoalkyl having 1 to 5 halogen atoms.

U represents a benzoheterocycle of the general formula (U-17)

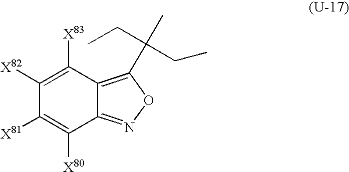

in which:

X⁸⁰, X⁸¹, X⁸² and X⁸³ may be the same or different and may be a hydrogen atom, a halogen atom, a hydroxyl group, an amino group, a nitro group, a C₁-C₄-alkyl, a C₁-C₄-alkoxy group, a C₁-C₄-alkylthio, a C₁-C₄-alkylsulphonyl or a C₁-C₄-halogenoalkyl having 1 to 5 halogen atoms.

U represents a benzoheterocycle of the general formula (U-18)

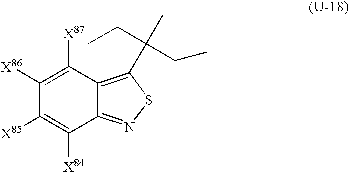

in which:

X⁸⁴, X⁸⁵, X⁸⁶ and X⁸⁷ may be the same or different and may be a hydrogen atom, a halogen atom, a hydroxyl group, an amino group, a nitro group, a C₁-C₄-alkyl, a C₁-C₄-alkoxy group, a C₁-C₄-alkylthio, a C₁-C₄-alkylsulphonyl or a C₁-C₄-halogenoalkyl having 1 to 5 halogen atoms.

U represents a benzoheterocycle of the general formula (U-19)

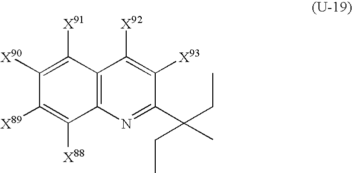

in which:

X⁸⁸, X⁸⁹, X⁹⁰, X⁹¹, X⁹² and X⁹³ may be the same or different and may be a hydrogen atom, a halogen atom, a hydroxyl group, an amino group, a nitro group, a C₁-C₄-alkyl, a C₁-C₄-alkoxy group, a C₁-C₄-alkylthio, a C₁-C₄-alkylsulphonyl or a C₁-C₄-halogenoalkyl having 1 to 5 halogen atoms.

U represents a benzoheterocycle of the general formula (U-20)

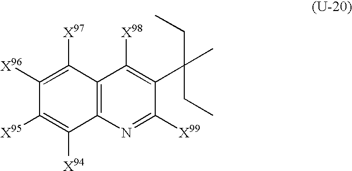

in which:

X⁹⁴, X⁹⁵, X⁹⁶, X⁹⁷, X⁹⁸ and X⁹⁹ may be the same or different and may be a hydrogen atom, a halogen atom, a hydroxyl group, an amino group, a nitro group, a C₁-C₄-alkyl, a C₁-C₄-alkoxy group, a C₁-C₄-alkylthio, a C₁-C₄-alkylsulphonyl or a C₁-C₄-halogenoalkyl having 1 to 5 halogen atoms.

U represents a benzoheterocycle of the general formula (U-21)

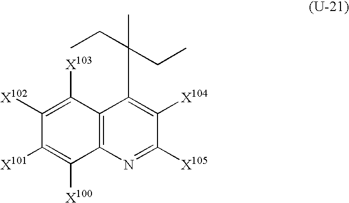

in which:

X¹⁰⁰, X¹⁰¹, X¹⁰², X¹⁰³, X¹⁰⁴ and X¹⁰⁵ may be the same or different and may be a hydrogen atom, a halogen atom, a hydroxyl group, an amino group, a nitro group, a C₁-C₄-alkyl, a C₁-C₄-alkoxy group, a C₁-C₄-alkylthio, a C₁-C₄-alkylsulphonyl or a C₁-C₄-halogenoalkyl having 1 to 5 halogen atoms.

U represents a benzoheterocycle of the general formula (U-22)

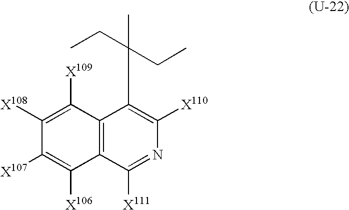

in which:

X¹⁰⁶, X¹⁰⁷, X¹⁰⁸, X¹⁰⁹, X¹¹⁰ and X¹¹¹ may be the same or different and may be a hydrogen atom, a halogen atom, a hydroxyl group, an amino group, a nitro group, a C₁-C₄-alkyl, a C₁-C₄-alkoxy group, a C₁-C₄-alkylthio, a C₁-C₄-alkylsulphonyl or a C₁-C₄-halogenoalkyl having 1 to 5 halogen atoms.

U represents a benzoheterocycle of the general formula (U-23)

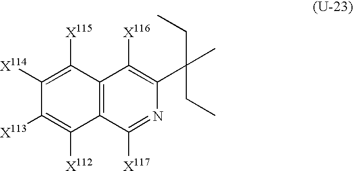

in which:

X¹¹², X¹¹³, X¹¹⁴, X¹¹⁵, X¹¹⁶ and X¹¹⁷ may be the same or different and may be a hydrogen atom, a halogen atom, a hydroxyl group, an amino group, a nitro group, a C₁-C₄-alkyl, a C₁-C₄-alkoxy group, a C₁-C₄-alkylthio, a C₁-C₄-alkylsulphonyl or a C₁-C₄-halogenoalkyl having 1 to 5 halogen atoms.

U represents a benzoheterocycle of the general formula (U-24)

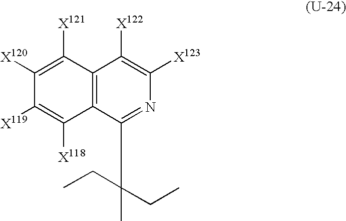

in which:

X¹¹⁸, X¹¹⁹, X², X¹²¹, X²² and X¹²³ may be the same or different and may be a hydrogen atom, a halogen atom, a hydroxyl group, an amino group, a nitro group, a C₁-C₄-alkyl, a C₁-C₄-alkoxy group, a C₁-C₄-alkylthio, a C₁-C₄-alkylsulphonyl or a C₁-C₄-halogenoalkyl having 1 to 5 halogen atoms.

The present invention also relates to a process for the preparation of the compound of general formula (I). Thus, according to a further aspect of the present invention there is provided a process for the preparation of a compound of general formula (I) as defined above, which comprises reacting a benzoheterocyclylethylamine derivative of general formula (II) or one of its salt:

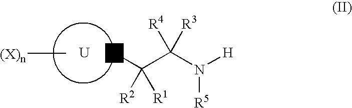

in which U, X, n, Z, R¹, R², R³, R⁴ and R⁵ are as defined above; with a carboxylic acid derivative of the general formula (III)

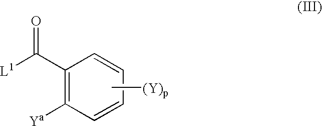

in which:

Y^{a}, Y and p are as defined above; and

L¹ is a leaving group chosen as being a halogen atom, a hydroxyl group, —OR⁶, —OCOR⁶, R⁶ being a C₁-C₆ alkyl, a C₁-C₆ haloalkyl, a benzyl, 4-methoxybenzyl, pentafluorophenyl or a group of formula

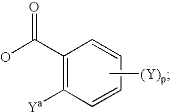

in the presence of a catalyst and, if L¹ is a hydroxyl group, in the presence of a condensing agent.

The process according to the present invention is conducted in the presence of a catalyst. Suitable catalyst may be chosen as being 4-dimethyl-aminopyridine, 1-hydroxy-benzotriazole or dimethylformamide.

In case L¹ is a hydroxy group, the process according to the present invention is conducted in the presence of a condensing agent. Suitable condensing agent may be chosen as being acid halide former, such as phosgene, phosphorous tribromide, phosphorous trichloride, phosphorous pentachloride, phosphorous trichloride oxide or thionyl chloride; anhydride former, such as ethyl chloroformate, methyl chloroformate, isopropyl chloroformate, isobutyl chloroformate or methanesulfonyl-chloride; carbodiimides, such as N,N′-dicyclohexylcarbodiimide (DCC), PS-DCC or other customary condensing agents, such as phosphorous pentoxide, polyphosphoric acid, N,N′-carbonyl-diimidazole, 2-ethoxy-N-ethoxycarbonyl-1,2-dihydroquinoline (EEDQ), triphenylphosphine/tetrachloromethane, 4-(4,6-dimethoxy[1.3.5]triazin-2-yl)-4-methylmorpholinium chloride hydrate or bromo-tripyrrolidino-phosphonium-hexafluorophosphate, 1-Hydroxybenzotriazole (HOBT).

When R⁵ is a hydrogen atom, the above mentioned process for the preparation of compound of general formula (I) may optionally be completed by a further step according to the following reaction scheme:

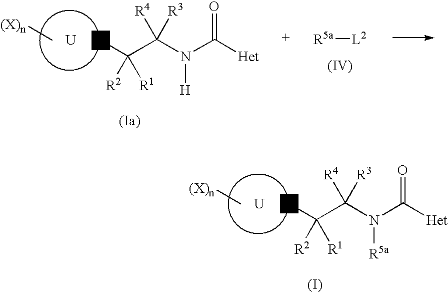

in which:
U, X, n, Z, R¹, R², R³, R⁴ and Het are as defined above;
R^{5a} is a C₁-C₆-alkyl, or a C₃-C₇-cycloalkyl;
L² is a leaving group chosen as being a halogen atom, a 4-methyl phenylsulfonyloxy or a methylsulfonyloxy;
   comprising the reaction of a compound of general formula (Ia) with a compound of general formula (IV) to provide a compound of general formula (I).

Depending on the definition of R¹, R², R³, R⁴ or R⁵, amine derivatives of general formula (II) may be prepared by different processes. One example (A) of such a process may be when:

U, X, n, z and R⁵ are as defined above;

R¹ is a hydrogen atom or a C₁-C₆ alkyl;

R² is a hydrogen atom or a C₁-C₆ alkyl; and

R³ and R⁴ are hydrogen atoms; then, the amine derivative of general formula (II) may be prepared according to a process which comprises:

a first step according to reaction scheme A-11

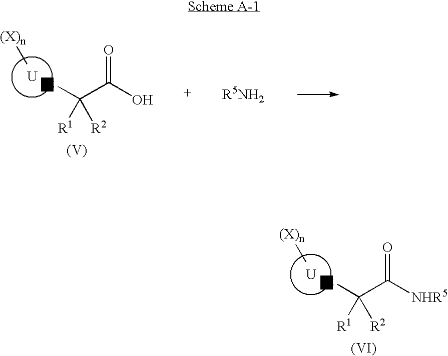

in which:

U, X, n, z and R⁵ are as defined above;

R¹ is a hydrogen atom or a C₁-C₆ alkyl; and

R² is a hydrogen atom or a C₁-C₆ alkyl; comprising the transformation of an benzoheterocyclic carboxylic acid of general formula (V) to provide an benzoheterocyclyl-amide derivative of general formula (VI), in the presence of an amine derivative and a condensing agent, at a temperature of from 0° C. to 200° C.;

a second step according to reaction scheme A-2:

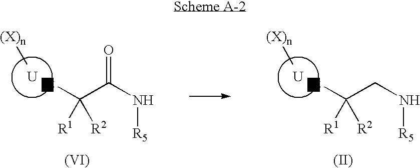

in which:

U, X, n, z and R⁵ are as defined above;

R¹ is a hydrogen atom or a C₁-C₆ alkyl; and

R² is a hydrogen atom or a C₁-C₆ alkyl; comprising a reduction of a compound of general formula (VI) to provide, a benzoheterocyclylethylamine derivative of general formula (II), in the presence of a hydride donor, at a temperature of from 0° C. to 200° C.;

The first step (step A-1) is conducted in the presence of a condensing agent. Suitable condensing agent may be chosen as being acid halide former, such as phosgene, phosphorous tribromide, phosphorous trichloride, phosphorous pentachloride, phosphorous trichloride oxide or thionyl chloride; sulfuryl chloride; anhydride former, such as ethyl chloroformate, methyl chloroformate, isopropyl chloroformate, isobutyl chloroformate or methanesulfonyl-chloride.

The first step (step A-1) according to the present invention is conducted at a temperature of from 0° C. to 200° C. Preferably, first step (step A-1) is conducted at a temperature of from 0° C. to 120° C.

The first step (step A-1) according to the present invention may be conducted in the presence of a solvent or not. When a solvent is used, the solvent is chosen preferably as being water, an organic solvent or a mixture of both. Suitable organic solvents may for example be aliphatic, alicyclic or aromatic solvent.

The second step (step A-2) according to the present invention is conducted in the presence of a hydride donor. Preferably, the hydride donor is chosen as being metal or metalloid hydrides such as LiAlH₄, NaBH₄, KBH₄, B₂H₆.

The second step (step A-2) according to the present invention may be conducted in the presence of a catalyst. Preferably, the catalyst is chosen as being a Lewis acid such as TiCl₄, AlCl₃, BF₃.Et₂O, CeCl₃.

The second step (step A-2) according to the present invention is conducted at a temperature of from 0° C. to 200° C. Preferably the temperature is of from 10° C. to 120° C. More preferably, the temperature is of from 10° C. to 80° C.

The second step (step A-2) according to the present invention may be conducted in the presence of an organic solvent, of water or of a mixture thereof. Preferably, the solvent is chosen as being ether, alcohol, carboxylic acid, or a mixture thereof with water or pure water.

When R⁵ is a hydrogen atom, the amine derivative of general formula (II) may also be prepared according to a process which comprises:

a first step according to reaction scheme B-1:

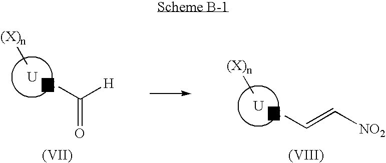

in which: —X, U, z and n are as defined above; comprising the transformation of an benzoheterocyclic aldehyde of general formula (VII) to provide an benzoheterocyclyl-nitroalkene derivative of general formula (VIII), at a temperature of from 0° C. to 200° C.;

a second step according to reaction scheme B-2:

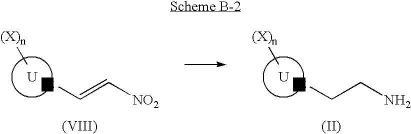

in which: —X, U, z and n are as defined above; comprising a reduction of a compound of general formula (VIII) to provide, a benzoheterocyclylethylamine derivative of general formula (II), in the presence of a hydride donor, at a temperature of from 0° C. to 200° C.;

The first step (step B-1) according to the present invention is conducted at a temperature of from 0° C. to 200° C. Preferably, first step (step A-1) is conducted at a temperature of from 0° C. to 150° C., more preferably at a temperature of from 50° C. to 150° C.

The first step (step B-1) according to the present invention may be conducted in the presence of an organic solvent, of water or of a mixture thereof. Preferably, the solvent is chosen as being ether, alcohol, carboxylic acid, or a mixture thereof with water or pure water.

The first step (step B-1) according to the present invention may be conducted in the presence of a buffer. Preferably the buffer is AcONH₄.

The second step (step B-2) according to the present invention is conducted in the presence of a hydride donor. Preferably, the hydride donor is chosen as being metal or metalloid hydrides such as LiAlH₄, NaBH₄, KBH₄, B₂H₆.

The second step (step B-2) according to the present invention is conducted at a temperature of from 0° C. to 200° C. Preferably the temperature is of from 10° C. to 120° C. More preferably, the temperature is of from 110° C. to 80° C.

The second step (step B-2) according to the present invention may be conducted in the presence of an organic solvent, of water or of a mixture thereof. Preferably, the solvent is chosen as being ether, alcohol, carboxylic acid, or a mixture thereof with water or pure water.

When R⁵ is a hydrogen atom, the amine derivative of general formula (II) may also be prepared according to a process which comprises:

a first step according to reaction scheme C-1:

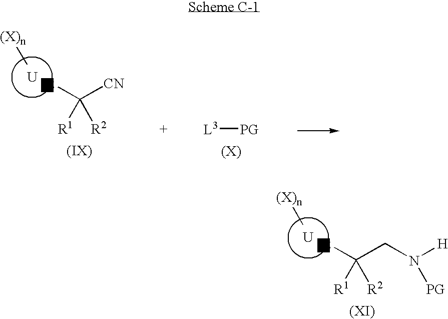

in which:
U, X, Z, n are as defined above;

R¹ is a hydrogen atom or a C₁-C₆ alkyl;
R² is a hydrogen atom or a C₁-C₆ alkyl;
L³ is a leaving group chosen as being a —OR⁷ group or a —OCOR⁷ group, R⁷ being a C₁-C₆ alkyl, a C₁-C₆ haloalkyl, a benzyl, 4-methoxybenzyl or pentafluorophenyl;
PG represents a protecting group which may be a —COOR⁷ group or —COR⁷ group, R⁷ being a C₁-C₆ alkyl, a C₁-C₆ haloalkyl, a benzyl, 4-methoxybenzyl or pentafluorophenyl; comprising the reduction, by hydrogenation or by an hydride donor, of a compound of general formula (IX), in the presence of a catalyst and in the presence of a compound of general formula (X) to produce a compound of general formula (X¹), at a temperature of from 0° C. to 150° C. and under a pressure of from 1 bar and 100 bar;

a second step according to reaction scheme C-2:

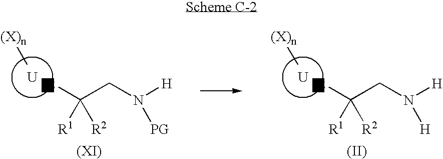

in which:
U, X, Z, n are as defined above;
R¹ is a hydrogen atom or a C₁-C₆ alkyl;
R² is a hydrogen atom or a C₁-C₆ alkyl
PG represents a protecting group which may be a —COOR⁷ group or —COR⁷ group, R⁷ being a C₁-C₆ alkyl, a C₁-C₆ haloalkyl, a benzyl, 4-methoxybenzyl or pentafluorophenyl;
   comprising a deprotection reaction, in an acidic or in a basic medium, of a compound of general formula (X¹) to provide an amine derivative of general formula (II) or one of its salt.

The compound according to the present invention can be prepared according to the general processes of preparation described above. It will nevertheless be understood that, on the basis of his general knowledge and of available publications, the skilled worker will be able to adapt this method according to the specifics of each of the compounds, which it is desired to synthesise.

On the basis of his general knowledge and of available publications, the skilled worker will also be able to prepare intermediate compounds of formula (V) (VII) and (IX) according to the present invention.

The present invention also relates to a benzoheterocyclylethylamine derivative of general formula (II) or one of its salt, and to its use as intermediate compound in the processes of preparation described above:

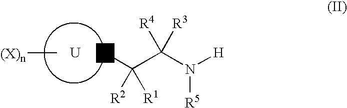

in which U, X, n, Z, R¹, R², R³, R⁴ and R⁵ are as defined above;

The present invention also relates to a fungicidal composition comprising an effective amount of an active material of general formula (I). Thus, according to the present invention, there is provided a fungicidal composition comprising, as an active ingredient, an effective amount of a compound of general formula (I) as defined above and an agriculturally acceptable support, carrier or filler.

In the present specification, the term “support” denotes a natural or synthetic, organic or inorganic material with which the active material is combined to make it easier to apply, notably to the parts of the plant. This support is thus generally inert and should be agriculturally acceptable. The support may be a solid or a liquid. Examples of suitable supports include clays, natural or synthetic silicates, silica, resins, waxes, solid fertilisers, water, alcohols, in particular butanol, organic solvents, mineral and plant oils and derivatives thereof. Mixtures of such supports may also be used.

The composition may also comprise additional components. In particular, the composition may further comprise a surfactant. The surfactant can be an emulsifier, a dispersing agent or a wetting agent of ionic or non-ionic type or a mixture of such surfactants. Mention may be made, for example, of polyacrylic acid salts, lignosulphonic acid salts, phenolsulphonic or naphthalenesulphonic acid salts, polycondensates of ethylene oxide with fatty alcohols or with fatty acids or with fatty amines, substituted phenols (in particular alkylphenols or arylphenols), salts of sulphosuccinic acid esters, taurine derivatives (in particular alkyl taurates), phosphoric esters of polyoxyethylated alcohols or phenols, fatty acid esters of polyols, and derivatives of the above compounds containing sulphate, sulphonate and phosphate functions. The presence of at least one surfactant is generally essential when the active material and/or the inert support are water-insoluble and when the vector agent for the application is water. Preferably, surfactant content may be comprised between 5% and 40% by weight of the composition.

Optionally, additional components may also be included, e.g. protective colloids, adhesives, thickeners, thixotropic agents, penetration agents, stabilisers, sequestering agents. More generally, the active materials can be combined with any solid or liquid additive, which complies with the usual formulation techniques.

In general, the composition according to the invention may contain from 0.05 to 99% (by weight) of active material, preferably 10 to 70% by weight.

Compositions according to the present invention can be used in various forms such as aerosol dispenser, capsule suspension, cold fogging concentrate, dustable powder, emulsifiable concentrate, emulsion oil in water, emulsion water in oil, encapsulated granule, fine granule, flowable concentrate for seed treatment, gas (under pressure), gas generating product, granule, hot fogging concentrate, macrogranule, microgranule, oil dispersible powder, oil miscible flowable concentrate, oil miscible liquid, paste, plant rodlet, powder for dry seed treatment, seed coated with a pesticide, soluble concentrate, soluble powder, solution for seed treatment, suspension concentrate (flowable concentrate), ultra low volume (ulv) liquid, ultra low volume (ulv) suspension, water dispersible granules or tablets, water dispersible powder for slurry treatment, water soluble granules or tablets, water soluble powder for seed treatment and wettable powder.

These compositions include not only compositions which are ready to be applied to the plant or seed to be treated by means of a suitable device, such as a spraying or dusting device, but also concentrated commercial compositions which must be diluted before application to the crop.

The compounds of the invention can also be mixed with one or more insecticides, fungicides, bactericides, attractant acaricides or pheromones or other compounds with biological activity. The mixtures thus obtained have a broadened spectrum of activity. The mixtures with other fungicides are particularly advantageous. Examples of suitable fungicide mixing partners may be selected in the following lists:

B1) a compound capable to inhibit the nucleic acid synthesis like benalaxyl, benalaxyl-M, bupirimate, chiralaxyl, clozylacon, dimethirimol, ethirimol, furalaxyl, hymexazol, metalaxyl-M, ofurace, oxadixyl, oxolinic acid;

B2) a compound capable to inhibit the mitosis and cell division like benomyl, carbendazim, diethofencarb, fuberidazole, pencycuron, thiabendazole thiophanate-methyl, zoxamide;

B3) a compound capable to inhibit the respiration for example as CI-respiration inhibitor like diflumetorim;

as CII-respiration inhibitor like boscalid, carboxin, fenfuram, flutolanil, furametpyr, mepronil, oxycarboxine, penthiopyrad, thifluzamide;

as CIII-respiration inhibitor like azoxystrobin, cyazofamid, dimoxystrobin, enestrobin, famoxadone, fenamidone, fluoxastrobin, kresoxim-methyl, metominostrobin, orysastrobin, pyraclostrobin, picoxystrobin, trifloxystrobin;

B4) a compound capable of to act as an uncoupler like dinocap, fluazinam;

B5) a compound capable to inhibit ATP production like fentin acetate, fentin chloride, fentin hydroxide, silthiofam;

B6) a compound capable to inhibit AA and protein biosynthesis like andoprim, blasticidin-S, cyprodinil, kasugamycin, kasugamycin hydrochloride hydrate, mepanipyrim, pyrimethanil;

B7) a compound capable to inhibit the signal transduction like fenpiclonil, fludioxonil, quinoxyfen;

B8) a compound capable to inhibit lipid and membrane synthesis like chlozolinate, iprodione, procymidone, vinclozolin, pyrazophos, edifenphos, iprobenfos (IBP), isoprothiolane, tolclofos-methyl, biphenyl, iodocarb, propamocarb, propamocarb-hydrochloride;

B9) a compound capable to inhibit ergosterol biosynthesis like fenhexamid, azaconazole, bitertanol, bromuconazole, cyproconazole, diclobutrazole, difenoconazole, diniconazole, diniconazole-M, epoxiconazole, etaconazole, fenbuconazole, fluquinconazole, flusilazole, flutriafol, furconazole, furconazole-cis, hexaconazole, imibenconazole, ipconazole, metconazole, myclobutanil, paclobutrazol, penconazole, propiconazole, prothioconazole, simeconazole, tebuconazole, tetraconazole, triadimefon, triadimenol, triticonazole, uniconazole, voriconazole, imazalil, imazalil sulfate, oxpoconazole, fenarimol, flurprimidol, nuarimol, pyrifenox, triforine, pefurazoate, prochloraz, triflumizole, viniconazole, aldimorph, dodemorph, dodemorph acetate, fenpropimorph, tridemorph, fenpropidin, spiroxamine, naftifine, pyributicarb, terbinafine;

B10) a compound capable to inhibit cell wall synthesis like benthiavalicarb, bialaphos, dimethomorph, flumorph, iprovalicarb, polyoxins, polyoxorim, validamycin A;

B11) a compound capable to inhibit melanine biosynthesis like carpropamid, diclocymet, fenoxanil, phtalide, pyroquilon, tricyclazole;

B12) a compound capable to induce a host defence like acibenzolar-5-methyl, probenazole, tiadinil;

B13) a compound capable to have a multisite action like captafol, captan, chlorothalonil, copper preparations such as copper hydroxide, copper naphthenate, copper oxychloride, copper sulphate, copper oxide, oxine-copper and Bordeaux mixture, dichlofluanid, dithianon, dodine, dodine free base, ferbam, fluorofolpet, folpet, guazatine, guazatine acetate, iminoctadine, iminoctadine albesilate, iminoctadine triacetate, mancopper, mancozeb, maneb, metiram, metiram zinc, propineb, sulphur and sulphur preparations including calcium polysulphide, thiram, tolylfluanid, zineb, ziram;

B14) a compound selected in the following list: amibromdole, benthiazole, bethoxazin, capsimycin, carvone, chinomethionat, chloropicrin, cufraneb, cyflufenamid, cymoxanil, dazomet, debacarb, diclomezine, dichlorophen, dicloran, difenzoquat, difenzoquat methylsulphate, diphenylamine, ethaboxam, ferimzone, flumetover, flusulfamide, fosetyl-aluminium, fosetyl-calcium, fosetyl-sodium, fluopicolide, fluoroimide, hexachlorobenzene, 8-hydroxyquinoline sulfate, irumamycin, methasulphocarb, metrafenone, methyl isothiocyanate, mildiomycin, natamycin, nickel dimethyldithiocarbamate, nitrothal-isopropyl, octhilinone, oxamocarb, oxyfenthiin, pentachlorophenol and salts, 2-phenylphenol and salts, phosphorous acid and its salts, piperalin, propanosine-sodium, proquinazid, pyrroInitrine, quintozene, tecloftalam, tecnazene, triazoxide, trichlamide, zarilamid and 2,3,5,6-tetrachloro-4-(methylsulfonyl)-pyridine, N-(4-Chloro-2-nitrophenyl)-N-ethyl-4-methyl-benzenesulfonamide, 2-amino-4-methyl-N-phenyl-5-thiazolecarboxamide, 2-chloro-N-(2,3-dihydro-1,1,3-trimethyl-1H-inden-4-yl)-3-pyridincarboxamide, 3-[5-(4-chlorophenyl)-2,3-dimethylisoxazolidin-3-yl]pyridine, cis-1-(4-chlorophenyl)-2-(1H-1,2,4-triazole-1-yl)-cycloheptanol, methyl 1-(2,3-dihydro-2,2-dimethyl-1H-inden-1-yl)-1H-imidazole-5-carboxylate, 3,4,5-trichloro-2,6-pyridinedicarbonitrile, Methyl 2-[[[cyclopropyl[(4-methoxyphenyl)imino]methyl]thio]methyl]-.alpha.-(methoxymethylene)-benzeneacetate, 4-Chloro-alpha-propynyloxy-N-[2-[3-methoxy-4-(2-propynyloxy)phenyl]ethyl]-benzeneacetamide, (2S)-N-[2-[4-[[3-(4-chlorophenyl)-2-propynyl]oxy]-3-methoxyphenyl]ethyl]-3-methyl-2-[(methylsulfonyl)amino]-butanamide, 5-chloro-7-(4-methylpiperidin-1-yl)-6-(2,4,6-trifluorophenyl)[1,2,4]triazolo[1,5-a]pyrimidine, 5-chloro-6-(2,4,6-trifluorophenyl)-N-[(1R)-1,2,2-trimethylpropyl][1,2,4]triazolo[1,5-a]pyrimidin-7-amine, 5-chloro-N-[(1R)-1,2-dimethylpropyl]-6-(2,4,6-trifluorophenyl)[1,2,4]triazolo[1,5-a]pyrimidin-7-amine, N-[1-(5-bromo-3-chloropyridin-2-yl)ethyl]-2,4-dichloronicotinamide, N-(5-bromo-3-chloropyridin-2-yl)methyl-2,4-dichloronicotinamide, 2-butoxy-6-iodo-3-propyl-benzopyranon-4-one, N-{(Z)-[(cyclopropylmethoxy)imino][6-(difluoromethoxy)-2,3-difluorophenyl]methyl}-2-phenylacetamide, N-(3-ethyl-3,5,5-trimethyl-cyclohexyl)-3-formylamino-2-hydroxy-benzamide, 2-[[[[1-[3(1-Fluoro-2-phenylethyl)oxy]phenyl]ethylidene]amino]oxy]methyl]-alpha-(methoxyimino)-N-methyl-alphaE-benzeneacetamide, N-{2-[3-chloro-5-(trifluoromethyl)pyridin-2-yl]ethyl}-2-(trifluoromethyl)benzamide, N-(3′,4′-dichloro-5-fluorobiphenyl-2-yl)-3-(difluoromethyl)-1-methyl-1H-pyrazole-4-carboxamide, 2-(2-{[6-(3-chloro-2-methylphenoxy)-5-fluoropyrimidin-4-yl]oxy}phenyl)-2-(methoxyimino)-N-methylacetamide, 1-[(4-methoxyphenoxy)methyl]-2,2-dimethylpropyl-1H-imidazole-1-carboxylic acid, O-[1-[(4-methoxyphenoxy)methyl]-2,2-dimethylpropyl]-1H-imidazole-1-carbothioic acid.

The composition according to the invention comprising a mixture of a compound of formula (I) with a bactericide compound may also be particularly advantageous. Examples of suitable bactericide mixing partners may be selected in the following list: bronopol, dichlorophen, nitrapyrin, nickel dimethyldithiocarbamate, kasugamycin, octhilinone, furancarboxylic acid, oxytetracycline, probenazole, streptomycin, tecloftalam, copper sulphate and other copper preparations.

The fungicidal compositions of the present invention can be used to curatively or preventively control the phytopathogenic fungi of crops. Thus, according to a further aspect of the present invention, there is provided a method for curatively or preventively controlling the phytopathogenic fungi of crops characterised in that a fungicidal composition as hereinbefore defined is applied to the seed, the plant and/or to the fruit of the plant or to the soil in which the plant is growing or in which it is desired to grow.

The composition as used against phytopathogenic fungi of crops comprises an effective and non-phytotoxic amount of an active material of general formula (I).

The expression “effective and non-phytotoxic amount” means an amount of composition according to the invention which is sufficient to control or destroy the fungi present or liable to appear on the crops, and which does not entail any appreciable symptom of phytotoxicity for the said crops. Such an amount can vary within a wide range depending on the fungus to be controlled, the type of crop, the climatic conditions and the compounds included in the fungicidal composition according to the invention.

This amount can be determined by systematic field trials, which are within the capabilities of a person skilled in the art.

The method of treatment according to the present invention is useful to treat propagation material such as tubers or rhizomes, but also seeds, seedlings or seedlings pricking out and plants or plants pricking out. This method of treatment can also be useful to treat roots. The method of treatment according to the present invention can also be useful to treat the overground parts of the plant such as trunks, stems or stalks, leaves, flowers and fruits of the concerned plant.

Among the plants that can be protected by the method according to the present invention, mention may be made of cotton; flax; vine; fruit or vegetable crops such as Rosaceae sp. (for instance pip fruit such as apples and pears, but also stone fruit such as apricots, almonds and peaches), Ribesioidae sp., Juglandaceae sp., Betulaceae sp., Anacardiaceae sp., Fagaceae sp., Moraceae sp., Oleaceae sp., Actimidaceae sp., Lauraceae sp., Musaceae sp. (for instance banana trees and plantins), Rubiaceae sp., Theaceae sp., Sterculiceae sp., Rutaceae sp. (for instance lemons, oranges and grapefruit); Solanaceae sp. (for instance tomatoes), Liliaceae sp., Asteraceae sp. (for instance lettuces), Umbelliferae sp., Cruciferae sp., Chenopodiaceae sp., Cucurbitaceae sp., Papilionaceae sp. (for instance peas), Rosaceae sp. (for instance strawberries); major crops such as Graminae sp. (for instance maize, lawn or cereals such as wheat, rice, barley and triticale), Asteraceae sp. (for instance sunflower), Cruciferae sp. (for instance colza), Fabacae sp. (for instance peanuts), Papilionaceae sp. (for instance soybean), Solanaceae sp. (for instance potatoes), Chenopodiaceae sp. (for instance beetroots); horticultural and forest crops; as well as genetically modified homologues of these crops.

Among the diseases of plants or crops that can be controlled by the method according to the present invention, mention may be made of:

Powdery mildew diseases such as:
Blumeria diseases, caused for example by Blumeria graminis;
Podosphaera diseases, caused for example by Podosphaera leucotricha;
Sphaerotheca diseases, caused for example by Sphaerotheca fuliginea;
Uncinula diseases, caused for example by Uncinula necator;
   Rust diseases such as:
Gymnosporangium diseases, caused for example by Gymnosporangium sabinae;
Hemileia diseases, caused for example by Hemileia vastatrix;
Phakopsora diseases, caused for example by Phakopsora pachyrhizi or Phakopsora meibomiae;
Puccinia diseases, caused for example by Puccinia recondita;
Uromyces diseases, caused for example by Uromyces appendiculatus;
   Oomycete diseases such as:
Bremia diseases, caused for example by Bremia lactucae;
Peronospora diseases, caused for example by Peronospora pisi or P. brassicae;
Phytophthora diseases, caused for example by Phytophthora infestans;
Plasmopara diseases, caused for example by Plasmopara viticola;
Pseudoperonospora diseases, caused for example by Pseudoperonospora humuli or Pseudoperonospora cubensis;
Pythium diseases, caused for example by Pythium ultimum;
Leafspot, leaf blotch and leaf blight diseases such as:
Alternaria diseases, caused for example by Alternaria solani;
Cercospora diseases, caused for example by Cercospora beticola;
Cladiosporum diseases, caused for example by Cladiosporium cucumerinum;
Cochliobolus diseases, caused for example by Cochliobolus sativus;
Colletotrichum diseases, caused for example by Colletotrichum lindemuthanium;
Cycloconium diseases, caused for example by Cycloconium oleaginum;
Diaporthe diseases, caused for example by Diaporthe citri;
Elsinoe diseases, caused for example by Elsinoe fawcettii;
Gloeosporium diseases, caused for example by Gloeosporium laeticolor;
Glomerella diseases, caused for example by Glomerella cingulata;
Guignardia diseases, caused for example by Guignardia bidwehi;
Leptosphaeria diseases, caused for example by Leptosphaeria maculans; Leptosphaeria nodorum;
Magnaporthe diseases, caused for example by Magnaporthe grisea;
Mycosphaerella diseases, caused for example by Mycosphaerella graminicola; Mycosphaerella arachidicola; Mycosphaerella fijiensis;
Phaeosphaeria diseases, caused for example by Phaeosphaeria nodorum;
Pyrenophora diseases, caused for example by Pyrenophora teres;
Ramularia diseases, caused for example by Ramularia collo-cygni;
Rhynchosporium diseases, caused for example by Rhynchosporium secalis;
Septoria diseases, caused for example by Septoria apii or Septoria lycopercisi;
Typhula diseases, caused for example by Typhula incarnata;
Venturia diseases, caused for example by Venturia inaequalis;
   Root and stem diseases such as:
Corticium diseases, caused for example by Corticium graminearum;
Fusarium diseases, caused for example by Fusarium oxysporum;
Gaeumannomyces diseases, caused for example by Gaeumannomyces graminis;
Rhizoctonia diseases, caused for example by Rhizoctonia solani;
Tapesia diseases, caused for example by Tapesia acuformis;
Thielaviopsis diseases, caused for example by Thielaviopsis basicola;
   Ear and panicle diseases such as:
Alternaria diseases, caused for example by Alternaria spp.;
Aspergillus diseases, caused for example by Aspergillus flavus;
Cladosporium diseases, caused for example by Cladosporium spp.;
Claviceps diseases, caused for example by Claviceps purpurea;
Fusarium diseases, caused for example by Fusarium culmorum;
Gibberella diseases, caused for example by Gibberella zeae;
Monographella diseases, caused for example by Monographella nivalis;
   Smut and bunt diseases such as:
Sphacelotheca diseases, caused for example by Sphacelotheca reiliana;
Tilletia diseases, caused for example by Tilletia caries;
Urocystis diseases, caused for example by Urocystis occulta;
Ustilago diseases, caused for example by Ustilago nuda;
   Fruit rot and mould diseases such as:
Aspergillus diseases, caused for example by Aspergillus flavus;
Botrytis diseases, caused for example by Botrytis cinerea;
Penicillium diseases, caused for example by Penicillium expansum;
Sclerotinia diseases, caused for example by Sclerotinia sclerotiorum;
Verticilium diseases, caused for example by Verticilium alboatrum;
   Seed and soilborne decay, mould, wilt, rot and damping-off diseases:
Fusarium diseases, caused for example by Fusarium culmorum;
Phytophthora diseases, caused for example by Phytophthora cactorum;
Pythium diseases, caused for example by Pythium ultimum;
Rhizoctonia diseases, caused for example by Rhizoctonia solani;
Sclerotium diseases, caused for example by Sclerotium rolfsii;
Microdochium diseases, caused for example by Microdochium nivale; Canker, broom and dieback diseases such as:
Nectria diseases, caused for example by Nectria galligena;
   Blight diseases such as:
Monilinia diseases, caused for example by Monilinia laxa; Leaf blister or leaf curl diseases such as:
Taphrina diseases, caused for example by Taphrina deformans;
   Decline diseases of wooden plants such as:
Esca diseases, caused for example by Phaemoniella clamydospora;
   Diseases of flowers and Seeds such as:
Botrytis diseases, caused for example by Botrytis cinerea;
   Diseases of tubers such as:
Rhizoctonia diseases, caused for example by Rhizoctonia solani.

The fungicide composition according to the present invention may also be used against fungal diseases liable to grow on or inside timber. The term “timber” means all types of species of wood, and all types of working of this wood intended for construction, for example solid wood, high-density wood, laminated wood, and plywood. The method for treating timber according to the invention mainly consists in contacting one or more compounds of the present invention, or a composition according to the invention; this includes for example direct application, spraying, dipping, injection or any other suitable means.

The dose of active material usually applied in the treatment according to the present invention is generally and advantageously between 10 and 800 g/ha, preferably between 50 and 300 g/ha for applications in foliar treatment. The dose of active substance applied is generally and advantageously between 2 and 200 g per 100 kg of seed, preferably between 3 and 150 g per 100 kg of seed in the case of seed treatment. It is clearly understood that the doses indicated above are given as illustrative examples of the invention. A person skilled in the art will know how to adapt the application doses according to the nature of the crop to be treated.

The fungicidal composition according to the present invention may also be used in the treatment of genetically modified organisms with the compounds according to the invention or the agrochemical compositions according to the invention. Genetically modified plants are plants into whose genome a heterologous gene encoding a protein of interest has been stably integrated. The expression “heterologous gene encoding a protein of interest” essentially means genes which give the transformed plant new agronomic properties, or genes for improving the agronomic quality of the transformed plant.

The compositions according to the present invention may also be used for the preparation of composition useful to curatively or preventively treat human and animal fungal diseases such as, for example, mycoses, dermatoses, trichophyton diseases and candidiases or diseases caused by Aspergillus spp., for example Aspergillus fumigatus.

The aspects of the present invention will now be illustrated with reference to the following tables of compounds and examples. The following Table illustrates in a non-limiting manner examples of fungicidal compounds according to the present invention. In the following Examples, M+1 (or M−1) means the molecular ion peak, plus or minus 1 a.m.u. (atomic mass units) respectively, as observed in mass spectroscopy and M (ApcI+) means the molecular ion peak as it was found via positive atmospheric pressure chemical ionisation in mass spectroscopy.

TABLE A

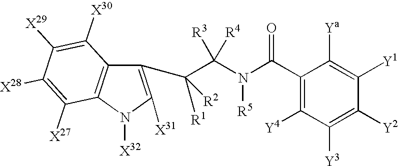

| Cmpd | R¹ | R² | R³ | R⁴ | R⁵ | Y^{a} | Y¹ | Y² | Y³ | Y⁴ | X²⁷ | X²⁸ | X²⁹ | X³⁰ | X³¹ | X³² | M + 1 |
|---|---|---|---|---|---|---|---|---|---|---|---|---|---|---|---|---|---|
| A-1 | H | H | H | H | H | CHF₂ | H | H | H | H | H | H | H | H | CH₃ | H | 329 |
| A-2 | H | H | H | H | H | CH₃ | H | H | H | H | H | H | H | H | CH₃ | H | 293 |
| A-3 | H | H | H | H | H | CF₃ | H | H | H | H | H | H | H | H | H | CH₃ | 347 |
| A-4 | H | H | H | H | H | CHF₂ | H | H | H | H | H | H | H | H | H | CH₃ | 329 |
| A-5 | H | H | H | H | H | I | H | H | H | H | H | H | OMe | H | H | CH₃ | 329 |
| A-6 | H | H | H | H | H | Br | H | H | H | H | H | H | OMe | H | H | CH₃ | 388 |
| A-7 | H | H | H | H | H | CF₃ | H | H | H | H | H | H | OMe | H | H | CH₃ | 435 |

TABLE B

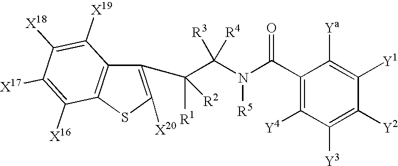

| Cmpd | R¹ | R² | R³ | R⁴ | R⁵ | Y^{a} | Y¹ | Y² | Y³ | Y⁴ | X¹⁶ | X¹⁷ | X¹⁸ | X¹⁹ | X²⁰ | M + 1 |
|---|---|---|---|---|---|---|---|---|---|---|---|---|---|---|---|---|
| B-1 | H | H | H | H | H | CHF₂ | H | H | H | H | H | H | Cl | H | H | 366 |
| B-2 | H | H | H | H | H | Br | H | H | H | H | H | H | Cl | H | H | 394 |
| B-3 | H | H | H | H | H | I | H | H | H | H | H | H | Cl | H | H | 442 |
| B-4 | H | H | H | H | H | CF₃ | H | H | H | H | H | H | Cl | H | H | 384 |
| B-5 | H | H | H | H | H | Br | H | H | H | H | H | H | H | H | H | 360 |
| B-6 | H | H | H | H | H | I | H | H | H | H | H | H | H | H | H | 408 |
| B-7 | H | H | H | H | H | CF₃ | H | H | H | H | H | H | H | H | H | 350 |

TABLE C

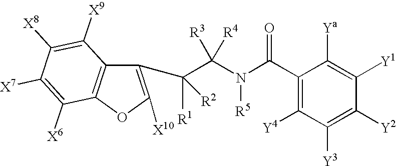

| Cmpd | R¹ | R² | R³ | R⁴ | R⁵ | Y^{a} | Y¹ | Y² | Y³ | Y⁴ | X⁶ | X⁷ | X⁸ | X⁹ | X¹⁰ | M + 1 |
|---|---|---|---|---|---|---|---|---|---|---|---|---|---|---|---|---|
| C-1 | H | H | H | H | H | Br | H | H | H | H | H | H | H | H | H | 344 |
| C-2 | H | H | H | H | H | I | H | H | H | H | H | H | H | H | H | 392 |
| C-3 | H | H | H | H | H | CF₃ | H | H | H | H | H | H | H | H | H | 334 |

TABLE D

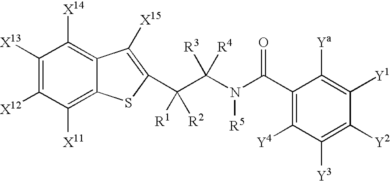

| Cmpd | R¹ | R² | R³ | R⁴ | R⁵ | Y^{a} | Y¹ | Y² | Y³ | Y⁴ | X¹¹ | X¹² | X¹³ | X¹⁴ | X¹⁵ | M + 1 |
|---|---|---|---|---|---|---|---|---|---|---|---|---|---|---|---|---|
| D-1 | H | H | H | H | H | CF₃ | H | H | H | H | H | H | H | H | CH₃ | 364 |
| D-2 | H | H | H | H | H | CF₃ | H | H | H | H | H | H | Cl | H | CH₃ | 398 |
| D-3 | H | H | H | H | H | I | H | H | H | H | H | H | H | H | CH₃ | 422 |
| D-4 | H | H | H | H | H | Br | H | H | H | H | H | H | H | H | CH₃ | 374 |
| D-5 | H | H | H | H | H | Br | H | H | H | H | H | H | Cl | H | CH₃ | 408 |
| D-6 | H | H | H | H | H | Cl | H | H | H | H | H | H | Cl | H | CH₃ | 364 |
| D-7 | H | H | H | H | H | Cl | H | H | H | H | H | H | H | H | CH₃ | 330 |
| D-8 | H | H | H | H | H | I | H | H | H | H | H | H | Cl | H | CH₃ | 456 |
| D-9 | H | H | H | H | H | CHF₂ | H | H | H | H | H | H | Cl | H | CH₃ | 380 |
| D-10 | H | H | H | H | H | CH₃ | H | H | H | H | H | H | H | H | CH₃ | 310 |
| D-11 | H | H | H | H | H | CHF₂ | H | H | H | H | H | H | H | H | CH₃ | 346 |

TABLE E

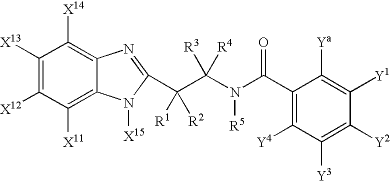

| Cmpd | R¹ | R² | R³ | R⁴ | R⁵ | Y^{a} | Y¹ | Y² | Y³ | Y⁴ | X¹¹ | X¹² | X¹³ | X¹⁴ | X¹⁵ | M + 1 |
|---|---|---|---|---|---|---|---|---|---|---|---|---|---|---|---|---|
| E-1 | Me | H | H | H | H | I | H | H | H | H | H | H | H | H | CH₃ | 362 |
| E-2 | Me | H | H | H | H | CF₃ | H | H | H | H | H | H | H | H | CH₃ | 420 |

TABLE F

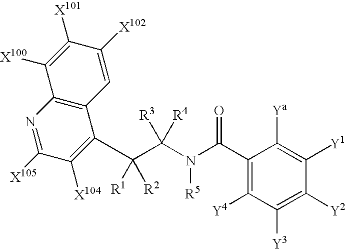

| Cmpd | R¹ | R² | R³ | R⁴ | R⁵ | Y^{a} | Y¹ | Y² | Y³ | Y⁴ | X¹⁰⁰ | X¹⁰¹ | X¹⁰² | X¹⁰³ | X¹⁰⁴ | X¹⁰⁵ | M + 1 |
|---|---|---|---|---|---|---|---|---|---|---|---|---|---|---|---|---|---|
| F-1 | H | H | H | H | H | CF₃ | H | H | H | H | H | H | H | H | H | H | 345 |

### Examples of Process for the Preparation of the Compound of General Formula (I)

### Synthesis of N-[2-(5-chloro-1-benzothien-3-yl)ethyl]-2-(trifluoromethyl)benzamide (Compound B-4)

### Preparation of tert-butyl [2-(5-chloro-1-benzothien-3-yl)ethyl]carbamate

To a stirred solution of (5-chloro-1-benzothien-3-yl)acetonitrile (5.30 g, 25.6 mmol), Nickel (II) chloride hexahydrate (6.05 g, 25.6 mmol), and di-tert-butyl dicarbonate (11.1 g, 51.0 mmol) in methanol (150 mL) under an inert atmosphere of nitrogen was added sodium borohydride (4.8 g, 126 mmol) in portions over a period of 10 min, maintaining the temperature below 45° C. The reaction mixture was stirred for 16 h and diluted with ethyl acetate (300 mL) and washed with brine (300 mL). The organic layer was dried (MgSO₄), filtered and concentrated in vacuo to afford an oil. Purification by flash chromatography using a 4:1 mixture of Heptane:Ethyl acetate afforded 7.8 g (98%) of tert-butyl [2-(5-chloro-1-benzothien-3-yl)ethyl]carbamate.

1HNMR (250 MHz, CDCl₃): δ 7.76-7.69 (2H, m); 7.36-7.15 (2H, m); 4.72-4.60 (N—H, b); 3.57-3.40 (2H, m); 3.1-2.95 (2H, m); 1.4 (9H, s).

### Preparation of 2-(5-chloro-1-benzothien-3-yloethanamine

A stirred solution of tert-butyl [2-(5-chloro-1-benzothien-3-yl)ethyl]carbamate (7.8 g, 25.1 mmol) in dichloromethane (100 mL) was treated with trifluoroacetic acid (28.5 g, 250 mmol) and the reaction mixture was stirred at RT for 16 h. An aqueous solution of sodium hydroxide (200 mL, 1N) was carefully added and the temperature was maintained below 20° C. The reaction mixture was stirred for 30 min and the organic layer was dried (MgSO₄), filtered and concentrated in vacuo to afford 4.9 g of the desired amine (92%).

1HNMR (250 MHz, CDCl₃): δ 7.82-7.70 (2H, m); 7.4-7.1 (2H, m); 3.16-2.90 (4H, m); 1.6-1.4 (NH₂, b).

The hydrochloride salt was obtained by addition of ethereal hydrochloric acid and filtration of the colorless precipitate.

### Synthesis of N-[2-(5-chloro-1-benzothien-3-yloethyl]-2-(trifluoromethyl)benzamide (Compound B-4)

A mixture of 2-(5-chloro-1-benzothien-3-yl)ethanamine (90.0 mg, 42.5 mmol) and triethylamine (43.5 mg, 42.5 mmol) in THF (5 mL) was treated with 2-trifluoromethylbenzoyl chloride (89.0 mg, 42.5 mmol) and the mixture was heated at reflux for 2 h. After cooling to RT, the mixture was concentrated in vacuo and the residue taken up in ethyl acetate (50 mL) and washed with water (50 mL). The organic layer was dried (MgSO₄), filtered and concentrated in vacuo. Purification of the residue by flash chromatography using a 4:1 mixture of Heptane:Ethyl acetate afforded 160 mg (94%) of N-[2-(5-chloro-1-benzothien-3-yl)ethyl]-2-(trifluoromethyl)benzamide (Compound B-4).

Mass Spectrum: [M+1]=384.

Examples of Biological Activity of the Compound of General Formula (I)

### EXAMPLE A

### In Vivo Test on Alternaria brassicae

The active ingredient tested is prepared by potter homogenisation in a concentrated suspension type formulation at 100 g/l. This suspension is then diluted with water to obtain the desired active material concentration.

Radish plants (Pernot variety) in starter cups, sown on a 50/50 peat soil-pozzolana substrate and grown at 18-20° C., are treated at the cotyledon stage by spraying with the aqueous suspension described above.

Plants, used as controls, are treated with an aqueous solution not containing the active material.

After 24 hours, the plants are contaminated by spraying them with an aqueous suspension of Alternaria brassicae spores (40,000 spores per cm³). The spores are collected from a 12-13-day-old culture.

The contaminated radish plants are incubated for 6-7 days at about 18° C., under a humid atmosphere.

Grading is carried out 6 to 7 days after the contamination, in comparison with the control plants.

Under these conditions, good (at least 50%) to total protection is observed at a dose of 330 ppm with the following compounds: D1, D2, D3 and D4 according to the invention and at a dose of 500 ppm with the following compounds: B1, B2, C3, D6, D8 and E1 according to the invention.

On the contrary, weak protection (less than 30%) to no protection at all is observed with N-benzoyltryptamine compounds. These N-benzoyltryptamine compounds are respectively:
N.[2-(5-bromo-1H-indol-3-yl)ethyl]-2-(trifluoromethyl)benzamide, tested at a dose of 330 ppm;
N.[2-(5-bromo-1H-indol-3-yl)ethyl]-2-chlorobenzamide, tested at a dose of 500 ppm;
N.[2-(5-bromo-1H-indol-3-yl)ethyl]-2-(difluoromethyl)benzamide, tested at a dose of 500 ppm.
   These results show that the compounds according to the invention have a much better biological activity than the structurally closest compounds disclosed in the Chinese patent application CN 1365971.

### EXAMPLE B

### In Vivo Test on Pyrenophora teres

The active ingredient tested is prepared by potter homogenisation in a concentrated suspension type formulation at 100 g/l. This suspension is then diluted with water to obtain the desired active material concentration.

Barley plants (Express variety) in starter cups, sown on a 50/50 peat soil-pozzolana substrate and grown at 12° C., are treated at the 1-leaf stage (10 cm tall) by spraying with the aqueous suspension described above. Plants, used as controls, are treated with an aqueous solution not containing the active material. After 24 hours, the plants are contaminated by spraying them with an aqueous suspension of Pyrenophora teres spores (12,000 spores per ml). The spores are collected from a 12-day-old culture. The contaminated barley plants are incubated for 24 hours at about 20° C. and at 100% relative humidity, and then for 12 days at 80% relative humidity.

Grading is carried out 12 days after the contamination, in comparison with the control plants.

Under these conditions, good (at least 50%) to total protection is observed at a dose of 330 ppm with the following compounds: D1 and D2 and at a dose of 500 ppm with the following compounds: C3, D3, D8 and F1. On the contrary, no protection at all is observed with N-benzoyltryptamine compounds. These N-benzoyltryptamine compounds are respectively:
N.[2-(5-bromo-1H-indol-3-yl)ethyl]-2-(trifluoromethyl)benzamide, tested at a dose of 330 ppm;
N.[2-(5-bromo-1H-indol-3-yl)ethyl]-2-chlorobenzamide, tested at a dose of 500 ppm;
N.[2-(5-bromo-1H-indol-3-yl)ethyl]-2-iodobenzamide, tested at a dose of 500 ppm;
N.[2-(5-bromo-1H-indol-3-yl)ethyl]-2-(difluoromethyl)benzamide, tested at a dose of 500 ppm.

Again, this confirm that the compounds according to the invention have a much better biological activity than the structurally closest compounds disclosed in the Chinese patent application CN 1365971.

1. A compound of general formula (I) 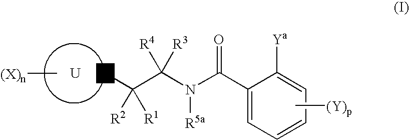
in which:
n is 0, 1, 2, 3, 4, 5 or 6;
p is 1, 2, 3 or 4;
U is: 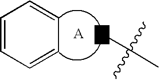
wherein A represents a 5- or 6-membered heterocycle including one, two or three heteroatoms which may be the same or different; fused with a phenyl ring to form a benzoheterocycle provided that this benzoheterocycle is different from 3-1H-2H-indolyl
z is a carbon atom or a heteroatom which can not be substituted by X;
X is the same or different and is a halogen atom, a nitro group, a cyano group, a hydroxy group, an amino group, a sulfanyl group, a pentafluoro-λ⁶-sulfanyl group, a formyl group, a formyloxy group, a formylamino group, a carboxy group, a carbamoyl group, a N-hydroxycarbamoyl group, a carbamate group, a (hydroxyimino)-C₁-C₆-alkyl group, a C₁-C₈-alkyl, a C₂-C₈-alkenyl, a C₂-C₈-alkynyl, a C₁-C₈-alkylamino, a C₁-C₈-halogenoalkyl having 1 to 5 halogen atoms, a di-C₁-C₈-alkylamino, a C₁-C₈-alkoxy, a C₁-C₈-halogenoalkoxy having 1 to 5 halogen atoms, a C₁-C₈-alkylsulfanyl, a C₁-C₈-halogenoalkylsulfanyl having 1 to 5 halogen atoms, a C₂-C₈-alkenyloxy, a C₂-C₈-halogenoalkenyloxy having 1 to 5 halogen atoms, a C₃-C₈-alkynyloxy, a C₃-C₈-halogenoalkynyloxy having 1 to 5 halogen atoms, a C₃-C₈-cycloalkyl, a C₃-C₈-halogenocycloalkyl having 1 to 5 halogen atoms, a C₁-C₈-alkylcarbonyl, a C₁-C₈-halogenoalkylcarbonyl having 1 to 5 halogen atoms, a C₁-C₈-alkylcarbamoyl, a di-C₁-C₈-alkylcarbamoyl, a N—C₁-C₈-alkyloxycarbamoyl, a C₁-C₈-alkoxycarbamoyl, a N—C₁-C₈-alkyl-C₁-C₈-alkoxycarbamoyl, a C₁-C₈-alkoxycarbonyl, a C₁-C₈-halogenoalkoxycarbonyl having 1 to 5 halogen atoms, a C₁-C₈-alkylcarbonyloxy, a C₁-C₈-halogenoalkylcarbonyloxy having 1 to 5 halogen atoms, a C₁-C₈-alkylcarbonylamino, a C₁-C₈-halogenoalkylcarbonylamino having 1 to 5 halogen atoms, a C₁-C₈-alkylaminocarbonyloxy, a di-C₁-C₈-alkylaminocarbonyloxy, a C₁-C₈-alkyloxycarbonyloxy, a C₁-C₈-alkylsulphenyl, a C₁-C₈-halogenoalkylsulphenyl having 1 to 5 halogen atoms, a C₁-C₈-alkylsulphinyl, a C₁-C₈-halogenoalkylsulphinyl having 1 to 5 halogen atoms, a C₁-C₈-alkylsulphonyl, a C₁-C₈-halogenoalkylsulphonyl having 1 to 5 halogen atoms, a C₁-C₆-alkoxyimino, a (C₁-C₆-alkoxyimino)-C₁-C₆-alkyl, a (C₁-C₆-alkenyloxyimino)-C₁-C₆-alkyl, a (C₁-C₆-alkynyloxyimino)-C₁-C₆-alkyl, a (benzyloxyimino)-C₁-C₆-alkyl, a benzyloxy, a benzylsulfanyl, a benzylamino, a phenoxy, a phenylsulfanyl a phenyl or a phenylamino;
R¹ and R² are the same or different and are a hydrogen atom, a cyano group, a formyl group, a carboxy group, a carbamoyl group, a (hydroxyimino)-C₁-C₆-alkyl group, a C₁-C₈-alkyl, a C₂-C₈-alkenyl, a C₂-C₈-alkynyl, a C₃-C₈-cycloalkyl, a C₃-C₈-halogenocycloalkyl having 1 to 5 halogen atoms, a C₁-C₈-alkylcarbonyl, a C₁-C₈-halogenoalkylcarbonyl having 1 to 5 halogen atoms, a C₁-C₈-halogenoalkyl having 1 to 5 halogen atoms, a C₁-C₈-alkylcarbamoyl, a di-C₁-C₈-alkylcarbamoyl, a C₁-C₈-alkoxycarbamoyl, a C₁-C₈-alkoxycarbonyl or a C₁-C₈-halogenoalkoxycarbonyl having 1 to 5 halogen atoms;
R³ and R⁴ are the same or different and are a hydrogen atom, a formyl group, a carbamoyl group, a (hydroxyimino)-C₁-C₆-alkyl group, a C₁-C₈-alkyl, a C₂-C₈-alkenyl, a C₂-C₈-alkynyl, a C₃-C₈-cycloalkyl, a C₃-C₈-halogenocycloalkyl having 1 to 5 halogen atoms, a C₁-C₈-alkylcarbonyl, a C₁-C₈-halogenoalkylcarbonyl having 1 to 5 halogen atoms, a C₁-C₈-halogenoalkyl having 1 to 5 halogen atoms, a C₁-C₈-alkylcarbamoyl, a di-C₁-C₈-alkylcarbamoyl or a C₁-C₈-alkoxycarbamoyl;
R⁵ is a hydrogen atom, a C₁-C₆-alkyl, or a C₃-C₇-cycloalkyl;
Y is the same or different and is a hydrogen atom, a halogen atom, a nitro group, a cyano group, a hydroxy group, an amino group, a sulfanyl group, a pentafluoro-1-sulfanyl group, a formyl group, a formyloxy group, a formylamino group, a carboxy group, a carbamoyl group, a N-hydroxycarbamoyl group, a carbamate group, a (hydroxyimino)-C₁-C₆-alkyl group, a C₁-C₈-alkyl, a C₂-C₈-alkenyl, a C₂-C₈-alkynyl, a C₁-C₈-alkylamino, a di-C₁-C₈-alkylamino, a C₁-C₉-alkoxy, a C₁-C₈-halogenoalkyl having 1 to 5 halogen atoms, a C₁-C₈-halogenoalkoxy having 1 to 5 halogen atoms, a C₁-C₈-alkylsulfanyl, a C₁-C₈-halogenoalkylsulfanyl having 1 to 5 halogen atoms, a C₂-C₈-alkenyloxy, a C₂-C₈-halogenoalkenyloxy having 1 to 5 halogen atoms, a C₃-C₈-alkynyloxy, a C₃-C₈-halogenoalkynyloxy having 1 to 5 halogen atoms, a C₃-C₈-cycloalkyl, a C₃-C₈-halogenocycloalkyl having 1 to 5 halogen atoms, a C₁-C₈-alkylcarbonyl, a C₁-C₈-halogenoalkylcarbonyl having 1 to 5 halogen atoms, a C₁-C₈-alkylcarbamoyl, a di-C₁-C₈-alkylcarbamoyl, a N—C₁-C₈-alkyloxycarbamoyl, a C₁-C₉-alkoxycarbamoyl, a N—C₁-C₈-alkyl-C₁-C₈-alkoxycarbamoyl, a C₁-C₈-alkoxycarbonyl, a C₁-C₈-halogenoalkoxycarbonyl having 1 to 5 halogen atoms, a C₁-C₈-alkylcarbonyloxy, a C₁-C₈-halogenoalkylcarbonyloxy having 1 to 5 halogen atoms, a C₁-C₈-alkylcarbonylamino, a C₁-C₈-halogenoalkylcarbonylamino having 1 to 5 halogen atoms, a C₁-C₈-alkylaminocarbonyloxy, a di-C₁-C₈-alkylaminocarbonyloxy, a C₁-C₈-alkyloxycarbonyloxy, a C₁-C₈-alkylsulphenyl, a C₁-C₈-halogenoalkylsulphenyl having 1 to 5 halogen atoms, a C₁-C₈-alkylsulphinyl, a C₁-C₈-halogenoalkylsulphinyl having 1 to 5 halogen atoms, a C₁-C₈-alkylsulphonyl, a C₁-C₈-halogenoalkylsulphonyl having 1 to 5 halogen atoms, a C₁-C₆-alkoxyimino, a (C₁-C₆-alkoxyimino)-C₁-C₆-alkyl, a (C₁-C₆-alkenyloxyimino)-C₁-C₆-alkyl, a (C₁-C₆-alkynyloxyimino)-C₁-C₆-alkyl or a (benzyloxyimino)-C₁-C₆-alkyl; and
Y^{a} is a halogen atom, a cyano group, a sulfanyl group, a pentafluoro-λ⁶-sulfanyl group, a formyl group, a formyloxy group, a formylamino group, a carbamoyl group, a N-hydroxycarbamoyl group, a carbamate group, a (hydroxyimino)-C₁-C₆-alkyl group, a C₁-C₈-alkyl, a C₂-C₈-alkenyl, a C₂-C₈-alkynyl, a C₁-C₈-alkoxy, a C₁-C₈-halogenoalkyl having 1 to 5 halogen atoms, a C₁-C₈-halogenoalkoxy having 1 to 5 halogen atoms, a C₁-C₈-alkylsulfanyl, a C₁-C₈-halogenoalkylsulfanyl having 1 to 5 halogen atoms, a C₂-C₈-alkenyloxy, a C₂-C₈-halogenoalkenyloxy having 1 to 5 halogen atoms, a C₃-C₈-alkynyloxy, a C₃-C₈-halogenoalkynyloxy having 1 to 5 halogen atoms, a C₃-C₈-cycloalkyl, a C₃-C₈-halogenocycloalkyl having 1 to 5 halogen atoms, a C₁-C₉-alkylcarbonyl, a C₁-C₈-halogenoalkylcarbonyl having 1 to 5 halogen atoms, a C₁-C₈-alkylcarbamoyl, a di-C₁-C₈-alkylcarbamoyl, a N—C₁-C₉-alkyloxycarbamoyl, a C₁-C₈-alkoxycarbamoyl, a N—C₁-C₈-alkyl-C₁-C₈-alkoxycarbamoyl, a C₁-C₈-alkoxycarbonyl, a C₁-C₈-halogenoalkoxycarbonyl having 1 to 5 halogen atoms, a C₁-C₈-alkylcarbonyloxy, a C₁-C₈-halogenoalkylcarbonyloxy having 1 to 5 halogen atoms, a C₁-C₈-alkylcarbonylamino, a C₁-C₈-halogenoalkylcarbonylamino having 1 to 5 halogen atoms, a C₁-C₈-alkylaminocarbonyloxy, a di-C₁-C₈-alkylaminocarbonyloxy, a C₁-C₈-alkyloxycarbonyloxy, a C₁-C₈-alkylsulphenyl, a C₁-C₈-halogenoalkylsulphenyl having 1 to 5 halogen atoms, a C₁-C₈-alkylsulphinyl, a C₁-C₈-halogenoalkylsulphinyl having 1 to 5 halogen atoms, a C₁-C₈-alkylsulphonyl, a C₁-C₈-halogenoalkylsulphonyl having 1 to 5 halogen atoms, a C₁-C₆-alkoxyimino, a (C₁-C₆-alkoxyimino)-C₁-C₆-alkyl, a (C₁-C₆-alkenyloxyimino)-C₁-C₆-alkyl or a (C₁-C₆-alkynyloxyimino)-C₁-C₆-alkyl, a (benzyloxyimino)-C₁-C₆-alkyl;
as well as its salts, N-oxydes, metallic complexes, metalloidic complexes and optically active isomers.

2. A compound according to claim 1, characterized in that n is 0, 1 or 2.

3. A compound according to claim 1, characterized in that X is chosen as being a C₁-C₄-alkyl group, a halogen atom, a hydroxyl group, an amino group, a nitro group, a C₁-C₄-alkoxy group, a C₁-C₄-alkylthio, a C₁-C₄-alkylsulphonyl, a C₁-C₄-halogenoalkyl having 1 to 5 halogen atoms.

4. A compound according to claim 1, characterized in that R¹ and R² are chosen, independently of each other, as being a hydrogen atom, a C₁-C₈-alkyl, a C₂-C₈-alkenyl, a C₂-C₈-alkynyl, a C₃-C₈-cycloalkyl, a C₃-C₈-halogenocycloalkyl having 1 to 5 halogen atoms, a C₁-C₈-halogenoalkyl having 1 to 5 halogen atoms.

5. A compound according to claim 1, characterized in that R³ and R⁴ are chosen, independently of each other, as being a hydrogen atom, a C₁-C₈-alkyl, a C₂-C₈-alkenyl, a C₂-C₈-alkynyl, a C₃-C₈-cycloalkyl, a C₃-C₈-halogenocycloalkyl having 1 to 5 halogen atoms, a C₁-C₈-halogenoalkyl having 1 to 5 halogen atoms.

6. A compound according to claim 1, characterized in that Y^{a} is chosen as being a halogen atom, a pentafluoro-λ⁶-sulfanyl group, a C₁-C₈-alkyl, a C₁-C₈-halogenoalkyl having 1 to 5 halogen atoms or a C₁-C₈-halogenoalkoxy having 1 to 5 halogen atoms.

7. A compound according to claim 1, characterized in that p is 1.

8. A compound according to claim 1, characterized in that Y is chosen as being a hydrogen atom or a halogen atom, preferably a hydrogen atom.

9. A compound according to claim 1, characterized in that U is chosen as being a 2-benzothiophene, a 3-benzothiophene, a 1-indole, a 2-indole, a 3-indole provided it is substituted in the 1- or in the 2-position, a 2-benzofuran, a 3-benzofuran, a 2-benzoxazole, a 2-benzothiazole, a 1-benzimidazole, a 2-benzimidazole, a 3-benz-1,2-isoxazole, a 3-benz-2,1-isoxazole, a 3-benz-1,2-isothiazole, a 3-benz-2,1-isothiazole, a 1-benzopyrazole, a 2-benzopyrazole, a 3-benzopyrazole, a 2-quinoline, a 3-quinoline, a 4-quinoline, a 1-isoquinoline, a 3-isoquinoline, a 4-isoquinoline.

10. A process for the preparation of a compound of general formula (I) as defined in claim 1, which comprises reacting a benzoheterocyclylethylamine derivative of general formula (II) or one of its salt: 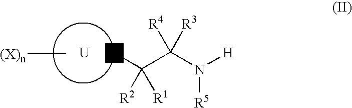
in which;
U is: 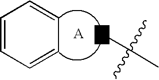
wherein A represents a 5- or 6-membered heterocycle including one, two or three heteroatoms which may be the same or different; fused with a phenyl ring to form a benzoheterocycle provided that this benzoheterocycle is different from 3-1H-2H-indolyl
Z is a carbon atom or a heteroatom which can not be substituted by X;
X is the same or different and is a halogen atom, a nitro group, a cyano group, a hydroxy group, an amino group, a sulfanyl group, a pentafluoro-λ⁶-sulfanyl group, a formyl group, a formyloxy group, a formylamino group, a carboxy group, a carbamoyl group, a N-hydroxycarbamoyl group, a carbamate group, a (hydroxyimino)-C₁-C₆-alkyl group, a C₁-C₈-alkyl, a C₂-C₈-alkenyl, a C₂-C₈-alkynyl, a C₁-C₈-alkylamino, a C₁-C₈-halogenoalkyl having 1 to 5 halogen atoms, a di-C₁-C₈-alkylamino, a C₁-C₈-alkoxy, a C₁-C₈-halogenoalkoxy having 1 to 5 halogen atoms, a C₁-C₈-alkylsulfanyl, a C₁-C₈-halogenoalkylsulfanyl having 1 to 5 halogen atoms, a C₂-C₈-alkenyloxy, a C₂-C₈-halogenoalkenyloxy having 1 to 5 halogen atoms, a C₃-C₈-alkynyloxy, a C₃-C₈-halogenoalkynyloxy having 1 to 5 halogen atoms, a C₃-C₈-cycloalkyl, a C₃-C₈-halogenocycloalkyl having 1 to 5 halogen atoms, a C₁-C₈-alkylcarbonyl, a C₁-C₈-halogenoalkylcarbonyl having 1 to 5 halogen atoms, a C₁-C₈-alkylcarbamoyl, a di-C₁-C₈alkylcarbamoyl, a N—C₁-C₈-alkyloxycarbamoyl, a C₁-C₈-alkoxycarbamoyl, a N—C₁-C₈-alkyl-C₁-C₈-alkoxycarbamoyl, a C₁-C₈-alkoxycarbonyl, a C₁-C₈-halogenoalkoxycarbonyl having 1 to 5 halogen atoms, a C₁-C₈-alkylcarbonyloxy, a C₁-C₈-halogenoalkylcarbonyloxy having 1 to 5 halogen atoms, a C₁-C₈-alkylcarbonylamino, a C₁-C₈-halogenoalkylcarbonylamino having 1 to 5 halogen atoms, a C₁-C₈-alkylaminocarbonyloxy, a di-C₁-C₈-alkylaminocarbonyloxy, a C₁-C₈-alkyloxycarbonyloxy, a C₁-C₈-alkylsulphenyl, a C₁-C₈-halogenoalkylsulphenyl having 1 to 5 halogen atoms, a C₁-C₈-alkylsulphinyl, a C₁-C₈-halogenoalkylsulphenyl having 1 to 5 halogen atoms a C₁-C₈-alkylsulphonyl, a C₁-C₈-halogenoalkylsulphonyl having 1 to 5 halogen atoms, a C₁-C₆-alkoxyimino, a (C₁-C₆-alkoxyimino)-C₁-C₆-alkyl, a (C₁-C₆-alkenyloxyimino)-C₁-C₆-alkyl, a (C₁-C₆-alkynyloxyimino)-C₁-C₆-alkyl, a (benzyloxyimino)-C₁-C₆-alkyl, a benzyloxy, a benzylsulfanyl, a benzylamino, a phenoxy, a phenylsulfanyl a phenyl or a phenylamino;
n is 0, 1, 2, 3, 4, 5 or 6;
R¹ and R² are the same or different and are a hydrogen atom, a cyano group, a formyl group, a carboxy group, a carbamoyl group, a (hydroxyimino)-C₁-C₆-alkyl group, a C₁-C₈-alkyl, a C₂-C₈-alkenyl, a C₂-C₈-alkynyl, a C₃-C₈-cycloalkyl, a C₃-C₈-halogenocycloalkyl having 1 to 5 halogen atoms, a C₁-C₈-alkylcarbonyl, a C₁-C₈-halogenoalkylcarbonyl having 1 to 5 halogen atoms, a C₁-C₈-halogenoalkyl having 1 to 5 halogen atoms, a C₁-C₈-alkylcarbamoyl, a di-C₁-C₈-alkylcarbamoyl, a C₁-C₈-alkoxycarbamoyl, a C₁-C₈-alkoxycarbonyl or a C₁-C₈ halogenoalkoxycarbonyl having 1 to 5 halogen atoms;
R³ and R⁴ are the same or different and are a hydrogen atom, a formyl group, a carbamoyl group, a (hydroxyimino)-C₁-C₆-alkyl group, a C₁-C₈-alkyl, a C₂-C₈-alkenyl, a C₂-C₈-alkynyl, a C₃-C₈-cycloalkyl, a C₃-C₈-halogenocycloalkyl having 1 to 5 halogen atoms, a C₁-C₈-alkylcarbonyl, a C₁-C₈-halogenoalkylcarbonyl having 1 to 5 halogen atoms, a C₁-C₈-halogenoalkyl having 1 to 5 halogen atoms, a C₁-C₈-alkylcarbamoyl, a di-C₁-C₈-alkylcarbamoyl or a C₁-C₈-alkoxycarbamoyl; and
R⁵ is a hydrogen atom, a C₁-C₆-alkyl, or a C₃-C₇-cycloalkyl;
with a carboxylic acid derivative of the general formula (III) 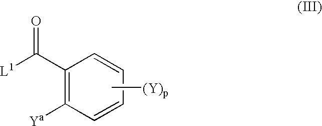
in which:
Y is the same or different and is a hydrogen atom, a halogen atom, a nitro group, a cyano group, a hydroxy group, an amino group, a sulfanyl group, a pentafluoro-λ⁶-sulfanyl group, a formyl group, a formyloxy group, a formylamino group, a carboxy group, a carbamoyl group, a N-hydroxycarbamoyl grout, a carbamate group, a (hydroxyimino)-C₁-C₆-alkyl group, a C₁-C₈-alkyl, a C₂-C₈-alkenyl, a C₂-C₈-alkynyl, a C₁-C₈-alkylamino, a di-C₁-C₈-alkylamino, a C₁-C₈-alkoxy, a C₁-C₉-halogenoalkyl having 1 to 5 halogen atoms, a C₁-C₈-halogenoalkoxy having 1 to 5 halogen atoms, a C₁-C₈-alkylsulfanyl, a C₁-C₈-halogenoalkylsulfanyl having 1 to 5 halogen atoms, a C₂-C₈-alkenyloxy, a C₂-C₈-halogenoalkenyloxy having 1 to 5 halogen atoms, a C₃-C₈-alkynyloxy, a C₃-C₈-halogenoalkynyloxy having 1 to 5 halogen atoms, a C₃-C₈-cycloalkyl, a C₃-C₈-halogenocycloalkyl having 1 to 5 halogen atoms, a C₁-C₈-alkylcarbonyl, a C₁-C₈-halogenoalkylcarbonyl having 1 to 5 halogen atoms, a C₁-C₈-alkylcarbamoyl, a di-C₁-C₈-alkylcarbamoyl, a N—C₁-C₈-alkyloxycarbamoyl, a C₁-C₈-alkoxycarbamoyl, a N—C₁-C₈-alkyl-C₁-C₈-alkoxycarbamoyl, a C₁-C₈-alkoxycarbonyl, a C₁-C₈-halogenoalkoxycarbonyl having 1 to 5 halogen atoms, a C₁-C₈-alkylcarbonyloxy, a C₁-C₈-halogenoalkylcarbonyloxy having 1 to 5 halogen atoms, a C₁-C₈-alkylcarbonylamino, a C₁-C₈-halogenoalkylcarbonylamino having 1 to 5 halogen atoms, a C₁-C₈-alkylaminocarbonyloxy, a di-C₁-C₈-alkylaminocarbonyloxy, a C₁-C₈-alkyloxycarbonyloxy, a C₁-C₈-alkylsulphenyl, a C₁-C₈-halogenoalkylsulphenyl having 1 to 5 halogen atoms, a C₁-C₈-alkylsulphinyl, a C₁-C₈-halogenoalkylsulphinyl having 1 to 5 halogen atoms, a C₁-C₈-alkylsulphonyl, a C₁-C₈-halogenoalkylsulphonyl having 1 to 5 halogen atoms, a C₁-C₆-alkoxyimino, a (C₁-C₆-alkoxyimino)-C₁-C₆-alkyl, a (C₁-C₆-alkenyloxyimino)-C₁-C₆-alkyl, a (C₁-C₆-alkynyloxyimino)-C₁-C₆-alkyl or a (benzyloxyimino)-C₁-C₆-alkyl;
Y^{a} is a halogen atom, a cyano group, a sulfanyl group, a pentafluoro-λ⁶-sulfanyl group, a formyl group, a formyloxy group, a formylamino group, a carbamoyl group, a N-hydroxycarbamoyl group, a carbamate group, a (hydroxyimino)-C₁-C₆-alkyl group, a C₁-C₈-alkyl, a C₂-C₈-alkenyl, a C₂-C₈-alkynyl, a C₁-C₈-alkoxy, a C₁-C₆-halogenoalkyl having 1 to 5 halogen atoms, a C₁-C₈-halogenoalkoxy having 1 to 5 halogen atoms, a C₂-C₈-alkylsulfanyl, a C₁-C₈-halogenoalkylsulfanyl having 1 to 5 halogen atoms, a C₂-C₈-alkenyloxy, a C₂-C₈-halogenoalkenyloxy having 1 to 5 halogen atoms, a C₃-C₈-alkynyloxy, a C₃-C₈ halogenoalkynyloxy having 1 to 5 halogen atoms, a C₃-C₈-cycloalkyl, a C₃-C₈-halogenocycloalkyl having 1 to 5 halogen atoms, a C₁-C₈-alkylcarbonyl, a C₁-C₈-halogenoalkylcarbonyl having 1 to 5 halogen atoms, a C₁-C₈-alkylcarbamoyl, a di-C₁-C₈-alkylcarbamoyl, a N—C₁-C₈-alkyloxycarbamoyl, a C₁-C₈-alkoxycarbamoyl, a N—C₁-C₈-alkyl-C₁-C₈-alkoxycarbamoyl, a C₁-C₈-alkoxycarbonyl, a C₁-C₈-halogenoalkoxycarbonyl having 1 to 5 halogen atoms, a C₁-C₈-alkylcarbonyloxy, a C₁-C₈-halogenoalkylcarbonyloxy having 1 to 5 halogen atoms, a C₁-C₈-alkylcarbonylamino, a C₁-C₈-halogenoalkylcarbonylamino having 1 to 5 halogen atoms, a C₁-C₈-alkylaminocarbonyloxy, a di-C₁-C₈-alkylaminocarbonyloxy, a C₁-C₈-alkyloxycarbonyloxy, a C₁-C₈-alkylsulphenyl, a C₁-C₈-halogenoalkylsulphenyl having 1 to 5 halogen atoms, a C₁-C₈-alkylsulphinyl, a C₁-C₈-halogenoalkylsulphinyl having 1 to 5 halogen atoms, a C₁-C₈-alkylsulphonyl, a C₁-C₈-halogenoalkylsulphonyl having 1 to 5 halogen atoms, a C₁-C₆-alkoxyimino, a (C₁-C₆-alkoxyimino)-C₁-C₆-alkyl, a (C₁-C₆-alkenyloxyimino)-C₁-C₆-alkyl or a (C₁-C₆-alkynyloxyimino)-C₁-C₆-alkyl, a (benzyloxyimino)-C₁-C₆-alkyl; and
p is 1, 2, 3 or 4; and
L¹ is a leaving group chosen as being a halogen atom, a hydroxyl group, —OR⁶, —OCOR⁶, R⁶ being a C₁-C₆ alkyl, a C₁-C₆ haloalkyl, a benzyl, 4-methoxybenzyl, pentafluorophenyl or a group of formula 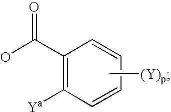
in the presence of a catalyst and, if L¹ is a hydroxyl group, in the presence of a condensing agent.

11. A process according to claim 10, characterized in that R⁵ is a hydrogen atom, and that the process is completed by a further step according to the following reaction scheme: 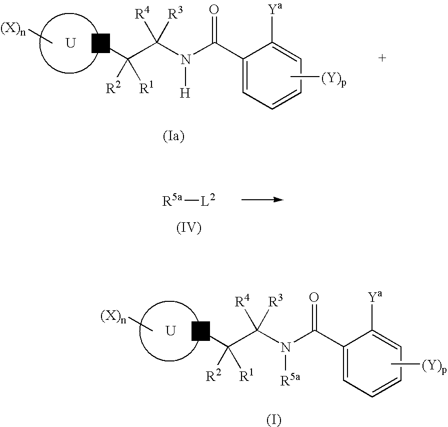
in which:
R^{5a} is a C₁-C₆-alkyl, or a C₃-C₇-cycloalkyl;
L² is a leaving group chosen as being a halogen atom, a 4-methyl phenylsulfonyloxy or a methylsulfonyloxy;
comprising the reaction of a compound of general formula (Ia) with a compound of general formula (IV) to provide a compound of general formula (I).

12. A compound of general formula (II) 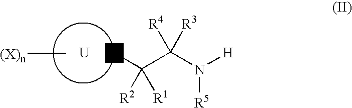
in which
U is: 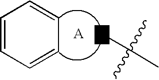
wherein A represents a 5- or 6-membered heterocycle including one, two or three heteroatoms which may be the same or different; fused with a phenyl ring to form a benzoheterocycle provided that this benzoheterocycle is different from 3-1H-2H-indolyl
Z is a carbon atom or a heteroatom which can not be substituted by X;
X is the same or different and is a halogen atom, a nitro group, a cyano group, a hydroxy group, an amino group, a sulfanyl group, a pentafluoro-λ⁶-sulfanyl group, a formyl group, a formyloxy group, a formylamino group, a carboxy group, a carbamoyl group, a N-hydroxycarbamoyl group, a carbamate group, a (hydroxyimino)-C₁-C₆-alkyl group a C₁-C₈-alkyl, a C₁-C₈-alkenyl, a C₁-C₈-alkynyl, a C₁-C₈-alkylamino, a C₁-C₈-halogenoalkyl having 1 to 5 halogen atoms, a di-C₁-C₈-alkylamino, a C₁-C₈-alkoxy, a C₁-C₈-halogenoalkoxy having 1 to 5 halogen atoms, a C₁-C₈-alkylsulfanyl, a C₁-C₈-halogenoalkylsulfanyl having 1 to 5 halogen atoms, a C₂-C₈-alkenyloxy, a C₂-C₈-halogenoalkenyloxy having 1 to 5 halogen atoms, a C₃-C₈-alkynyloxy, a C₁-C₈-halogenoalkynyloxy having 1 to 5 halogen atoms, a C₃-C₈-cycloalkyl, a C₃-C₈-halogenocycloalkyl having 1 to 5 halogen atoms, a C₁-C₈-alkylcarbonyl, a C₁-C₈-halogenoalkylcarbonyl having 1 to 5 halogen atoms, a C₁-C₈-alkylcarbamoyl, a di-C₁-C₈-alkylcarbamoyl, a N—C₁-C₈-alkyloxycarbamoyl, a C₁-C₈-alkoxycarbamoyl, a N—C₁-C₈-alkyl-C₁-C₈-alkoxycarbamoyl, a C₁-C₈-alkoxycarbonyl, a C₁-C₈-halogenoalkoxycarbonyl having 1 to 5 halogen atoms, a C₁-C₈-alkylcarbonyloxy, a C₁-C₈-halogenoalkylcarbonyloxy having 1 to 5 halogen atoms, a C₁-C₈-alkylcarbonylamino, a C₁-C₈-halogenoalkylcarbonylamino having 1 to 5 halogen atoms, a C₁-C₈-alkylaminocarbonyloxy, a di-C₁-C₈-alkylaminocarbonyloxy, a C₁-C₈-alkyloxycarbonyloxy, a C₁-C₈-alkylsulphenyl, a C₁-C₈-halogenoalkylsulphenyl having 1 to 5 halogen atoms, a C₁-C₈-alkylsulphinyl, a C₁-C₈-halogenoalkylsulphinyl having 1 to 5 halogen atoms, a C₁-C₈-alkylsulphonyl, a C₁-C₈-halogenoalkylsulphonyl having 1 to 5 halogen atoms, a C₁-C₆-alkoxyimino, a (C₁-C₆-alkoxyimino)-C₁-C₆-alkyl, a (C₁-C₆-alkenyloxyimino)-C₁-C₆-alkyl, a (C₁-C₆-alkynyloxyimino)-C₁-C₆-alkyl, a (benzyloxyimino)-C₁-C₆-alkyl, a benzyloxy, a benzylsulfanyl, a benzylamino, a phenoxy, a phenylsulfanyl a phenyl or a phenylamino;
n is 0, 1, 2, 3, 4, 5 or 6;
R¹ and R² are the same or different and are a hydrogen atom, a cyano group, a formyl group, a carboxy group, a carbamoyl group, a (hydroxyimino)-C₁-C₆-alkyl group, a C₁-C₈-alkyl, a C₂-C₈-alkenyl, a C₂-C₈-alkynyl, a C₃-C₈-cycloalkyl, a C₃-C₈halogenocycloalkyl having 1 to 5 halogen atoms, a C₁-C₈-alkylcarbonyl, a C₁-C₈-halogenoalkylcarbonyl having 1 to 5 halogen atoms, a C₁-C₈-halogenoalkyl having 1 to 5 halogen atoms, a C₁-C₈-alkylcarbamoyl, a di-C₁-C₈-alkylcarbamoyl, a C₁-C₈-alkoxycarbamoyl, a C₁-C₈-alkoxycarbonyl or a C₁-C₈-halogenoalkoxycarbonyl having 1 to 5 halogen atoms;
R³ and R⁴ are the same or different and are a hydrogen atom, a formyl group, a carbamoyl group, a (hydroxyimino)-C₁-C₆-alkyl group, a C₁-C₈-alkyl, a C₁-C₈-alkenyl, a C₂-C₈-alkynyl, a C₃-C₈-cycloalkyl, a C₃-C₈-halogenocycloalkyl having 1 to 5 halogen atoms, a C₁-C₈-alkylcarbonyl, a C₁-C₈-halogenoalkylcarbonyl having 1 to 5 halogen atoms, a C₁-C₈-halogenoalkyl having 1 to 5 halogen atoms, a C₁-C₈-alkylcarbamoyl, a di-C₁-C₈-alkylcarbamoyl or a C₁-C₈-alkoxycarbamoyl; and
R⁵ is a hydrogen atom, a C₁-C₆-alkyl, or a C₃-C₇-cycloalkyl.

13. Fungicide composition comprising an effective amount of a compound according to claim 1 and an agricultural acceptable support.

14. Method for preventively or curatively combating the phytopathogenic fungi of crops, characterized in that an effective and non-phytotoxic amount of a composition according to claim 13 is applied to the plant seeds or to the plant leaves and/or to the fruits of the plants or to the soil in which the plants are growing or in which it is desired to grow them.
